(19) United States
(12) Patent Application Publication
(10) Pub. No.: US 2023/0044427 A1
(43) Pub. Date: Feb. 9, 2023

(54) EVAPORATION RETARDANT MEMBRANE FOR ODORANT COMPOSITIONS

(71) Applicant: BASF SE, Ludwigshafen am Rhein (DE)

(72) Inventor: Ingmar BAUS, Otterstadt (DE)

(21) Appl. No.: 17/793,423

(22) PCT Filed: Jan. 19, 2021
(86) PCT No.: PCT/EP2021/051057

§ 371 Date: Jul. 18, 2022

(30) Foreign Application Priority Data
Jan. 20, 2020 (EP) 20152677.9

(51) Int. Cl. A61L 9/04

(52) U.S. Cl. CPC A61L 9/042; A61L 2209/131

(57) ABSTRACT

The present invention relates to an evaporation retardant membrane for odorant compositions, which comprises a sheet-like support permeable to odorants, and at least one polymer coating arranged on one surface of the sheet-like support, wherein the sheet-like support is permeable to fragrances and where on at least one surface of the sheet-like support at least one polymer coating is arranged only on a part of the surface of the sheet-like support, such that the total coverage of the surface by all polymer coatings is 10 to 90%, and each quarter square centimeter of said surface is covered by a polymer coating to an extent of at least 10%. The present invention also relates to a device for controlled release of an odorant composition comprising a container for receiving an odorant composition, where the container has an opening, which is covered by the evaporation retardant membrane and the use of such a device for controlled release of odorant compositions.

The present invention relates to an evaporation retardant membrane for odorant compositions, which comprises a sheet-like support permeable to odorants, and at least one polymer coating arranged on one surface of the sheet-like support. The present invention also relates to a device for controlled release of an odorant composition comprising a container for receiving an odorant composition, where the container has an opening, which is covered by the evaporation retardant membrane and the use of such a device for controlled release of odorant compositions.

The controlled time release of volatile substances, such as odorants and other aromatic products, presents a number of packaging problems. Typically, devices for controlled time release of volatile substances comprise a container for receiving the volatile substance, such as odorant compositions, and means for controlled release of the volatile substance to the environment, such as ambient air.

A particular problem associated with controlled time release of volatile odorant substances is that the more volatile components of the odorant composition evaporate faster than the less volatile components of the composition. This will lead to a drastic shift of the odorant composition and results in a noticeable olfactory profile change of the air freshener product during its use. Especially citrus, fruity and other fresh perfume types suffer a dramatic change from fresh, citrus like smell at the beginning of product use to a more mellow smell of typical heart and base notes, such as woody, musk and some heavy floral smells. Faster evaporation of an odorant composition’s top note occurs due to its higher volatility. During usage of conventional refresheners the top note will exhaust itself faster than the slower evaporating heart and bottom notes. This effect is undesirable since the consumer expects the product to maintain its olfactory character throughout the lifetime of the product.

A very simple embodiment of devices for controlled time release of volatile substances are conventional room “air fresheners”. In these devices, the odorant composition is contained in a glass bottle or vial. The odorant is released into the atmosphere by transmission through an absorbent wick or a wooden stick. The bottle is usually capped until the time of use. During use, the bottle or vial is usually not completely closed and, therefore, the odorant may be released in an uncontrolled manner. Apart from that, these devices do not solve the problem of different evaporation rates.

Other commercial devices for controlled time release comprise a container filled with volatile substance closed with a membrane through which the volatile diffuses and bys, which it is thereby released to the ambient air. Typically, the membrane is a foil made of synthetic polymers, such as polypropylene PP or polyethylene PE, ethylene-vinyl acetate copolymers EVA, copolymers of ethylene and alkyl acrylates or polyethylene terephthalate PET. The foil may be monolithic or microporous. Unfortunately, the diffusion rate of the different volatile compounds of typical odorant compositions will depend not only on the different vapor pressures of the volatile compounds in the composition but also on the chemical nature of the polymer, which forms the membrane, in particular its polarity and its glass transition temperature, and on the different polarities of the volatile compounds. Therefore, the overall composition of the odorant in the container will change during use, which leads to a pronounced change of olfactory character of the product.

It is also known to control the release of odorants by including the odorant substance into a gel formulation. However, the gel must be adapted to the odorant compounds to achieve a controlled release and it is difficult and not always possible to provide a gel, which is suitable for achieving the desired control.

US 2001/000235 describes a permeable membrane for volatile substances such as odorants and other aroma chemicals. The membrane is part of a multilayer structure, which comprises at least two different permeable layers, which form a membrane, and a release layer, which is impermeable and can be removed from the permeable layers. Upon removal of the release layer, the membrane layers are exposed to the ambient air and the odorants diffuse through the membrane layer and are released to the ambient air. The multilayer is produced by multistep cast extrusion of the different permeable and release layers, which is quite tedious. Moreover, these membranes typically require more than two membrane layers in order to achieve a uniform release of the different components of an odorant composition.

JPH 08164193 describes a fragrance dispenser comprising cup-shaped container body for receiving the perfume, which is closed by a permeable membrane adhered to the flange portions of the container. The permeable membrane is formed from a non-woven, a paper or a synthetic paper and covered by a lid, which may be peeled off to start the release of the perfume. While the membrane slows the release of the perfume, it does not achieve a uniform release.

WO 2008/104226 describes a fragrance dispenser for controlled release of a combination of highly volatile fragrance substances HVA and low volatile fragrance substances LVA. In the dispenser, the HVA and the LVA are adsorbed in different solid carriers, which are spatially separated from each other. The release rate of the HVA is controlled by an evaporation retardant barrier, while the release of the LVA is not controlled. The construction of such a device is complex, and the different fragrance components must be formulated separately.

KR 20150059545 describes a fragrance dispenser for controlled release of a fragrance, which comprises a fragrance receiving unit, which is loaded with the fragrance and which is connected to a second absorbing unit, from which the fragrance is released to the environment, where an outflow control unit, such as micro-channels, is located between the fragrance receiving unit and said absorbing unit to control the release of the fragrance from the fragrance receiving unit to the said absorbing unit and thereby avoiding an uncontrolled release to the environment. The design of the dispenser is complex and therefore its production is quite expensive.

US 2017/000102 discloses a dispenser for liquid volatile substances comprising a reservoir containing the liquid volatile substance, a microporous membrane positioned over the reservoir, said membrane being affixed to the peripheral portion of the reservoir and having a barrier coating layer over the outer surface of the membrane, while the inner surface of the membrane contacts the liquid volatile substance. The dispenser also has a removable cap layer adhered by an adhesive layer to the outer surface of the microporous membrane such that the microporous vapor-permeable membrane and the liquid volatile substance are substantially sealed beneath the cap layer. The microporous membrane comprises a polymer matrix, an interconnecting network of pores communicating throughout the polymeric matrix, and a finely divided, substantially water-insoluble filler material. Such microporous membranes are difficult to produce and thus expensive.

There is still a demand for odorant release devices, which provide a pleasant, natural aroma over a prolonged period of time without significant changes of odorant strength and/or odorant character, in particular for odorant compositions containing at least two different odorant compounds, where the different odorant compounds have different polarities and/or different vapor pressures. Moreover, the devices should not require complex design, be easy to manufacture and have no expensive components

It was surprisingly found that partial coating of materials permeable for volatiles with a polymer coating allows for targeted or tailor-made permeation of aromas, fragrances or other similar odorants. While the polymer coating forms a retardant barrier for most of the volatile compounds, the fact that only parts of the permeable material are coated allows for an easy adjustment of the different diffusion rates of the different volatile compounds of an odorant composition. In particular, it is possible to tailor the relative release rate of the odorant components by the degree of surface covering, by the coating thickness, by the polarity of the polymer of the polymer coating, by the glass transition temperature of the polymer of the polymer coating and by combinations of these measures. In particular, it is possible to tailor the evaporation retardant membranes of the invention to the relative release rates of the odorant components by combining at least two different polymer coatings on the surface of the carrier.

Therefore, a first aspect of the present invention relates to the use of sheets comprising a sheet-like support and at least one polymer coating arranged on at least one surface of the sheet-like support as an evaporation retardant membrane for odorant compositions, wherein the sheet-like support is permeable to odorants and where on at least one of the surfaces of the support at least one polymer coating is arranged only on a part of the surface of the sheet-like support such that the total coverage of the surface by all polymer coatings is 10 to 90%, in particular 20 to 80%, and each quarter square centimeter of said surface is covered by a polymer coating to an extent of at least 10%, in particular at least 20%.

A second aspect of the present invention relates to a device for controlled release of an odorant composition comprising a container for receiving an odorant composition, where the container has an opening, which is covered by a membrane, which is an evaporation retardant membrane as defined herein.

The present invention is associated with several advantages. The evaporation retardant membranes of the invention allow for a more uniform release of the components of an odorant than conventional membranes, where the evaporation retardation is achieved by a full-surface polymer layer, such as a monolithic sheet or a microporous sheet. Moreover, evaporation retardant membranes of the invention can be easily produced by simple printing techniques. What is more, the evaporation retardant membranes of the invention can be easily adjusted to the different diffusion rates of the different volatile compounds usually contained in odorant compositions. In particular, the evaporation retardant membranes of the invention can be easily tailored to the relative release rates of the different odorant components, e.g. by the degree of surface covering, by the coating thickness, by the polarity of the polymer of the polymer coating, by the glass transition temperature of the polymer of the polymer coating and by combinations of these measures.

Here and in the following, the term “polymer coating” refers to a coating, comprising at least one organic polymer as a main constituent, i.e. the total amount of any organic polymer in the coating is at least 50% by weight, i.e. 50 to 100% by weight, based on the total mass of the polymer coating.

Here and in the following an X % coverage means that the respective surface area is covered to an extent of X %, based on said surface area.

Here and throughout the specification, the prefixes Cₙ-Cₘ used in connection with compounds or molecular moieties each indicate a range for the number of possible carbon atoms that a molecular moiety or a compound can have. The term “C₁-Cₙ alkyl” denominates a group of linear or branched saturated hydrocarbon radicals having from 1 to n carbon atoms. For example, the term C₁-C₂₀ alkyl denominates a group of linear or branched saturated hydrocarbon radicals having from 1 to 20 carbon atoms, while the terms C₁-C₁₀ alkyl and C₁-C₄ alkyl denominate a group of linear or branched saturated hydrocarbon radicals having from 1 to 10 carbon atoms or 1 to 4 carbon atoms, respectively. Examples of alkyl include but are not limited to methyl, ethyl, n-propyl, isopropyl, n-butyl, 2-butyl, 2-methylpropyl (isopropyl), 1,1-dimethylethyl (tert-butyl), pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, heptyl, octyl, 2-ethylhexyl, nonyl, decyl, undecyl, dodecyl, tridecyl, tetradecyl, pentadecyl, hexadecyl, heptadecyl, octadecyl, nonadecyl, eicosyl, heneicosyl docosyl and their isomers. Examples of C₁-C₄-alkyl are for example methyl, ethyl, propyl, 1-methylethyl, butyl, 1-methylpropyl, 2-methylpropyl or 1,1-dimethylethyl.

Here and throughout the specification, the term “(meth)acryl” includes both acryl and methacryl groups. Hence, the term “(meth)acrylate” includes acrylate and methacrylate, and the term “(meth)acrylamide” includes acrylamide and methacrylamide.

According to the invention, the polymer coating may be arranged on one or both surfaces, in particular on one surface of the sheet-like support. The coating covers only a part of said surface on which it is arranged, such that the total coverage of the surface by all polymer coatings, which are present on said surface, is 10 to 90%, in particular 20 to 80% of the total area of the surface, which forms the evaporation retardant membrane. Consequently, 10 to 90%, in particular 20 to 80% of the total area of said surface is not covered by any polymer coating. If both surfaces of the sheet-like support are covered by a polymer coating, it is sufficient that only one of the surfaces is only partly covered by the polymer coating. In other words, if both surfaces of the sheet-like support are covered by one or more polymer coatings, the total coverage of one surface by all polymer coatings, which are present on said surface, is 10 to 90%, in particular 20 to 80% of the total area of the surface, while the other surface may be completely covered by one or more polymer coatings. If both surfaces of the sheet-like support are covered by one or more polymer coatings, it is preferred that on both surfaces the total coverage of the respective surface by all polymer coatings, which are present on said surface, is 10 to 90%, in particular 20 to 80% of the total area of said surface. In particular, only one of the surfaces of the sheet-like support is partly covered by the polymer coating such that the total coverage of the surface by all polymer coatings, which are present on said surface, is 10 to 90%, in particular 20 to 80% of the total area of said surface.

In contrast to a covering, the polymer coating is permanently bonded to the surface of the sheet-like support coating and cannot be mechanically removed from the carrier without damaging the surface of the carrier or even destroying the carrier.

According to the invention, the polymer coating is evenly arranged on the respective surface, i.e. each quarter square centimeter of said surface is at least partly covered by the polymer coating to such an extent that the coverage of each quarter square centimeter is at least 10%, in particular at least 20% of the area of said quarter square centimeter. Preferably, each square centimeter of said surface of the sheet-like support is covered by a polymer coating to an extent of 10 to 90%, in particular 20 to 80% of the area of said quarter square centimeter. Consequently, at most 90%, in particular at most 80% of the area each quarter square centimeter are not covered by a polymer coating. In particular, at least 10%, especially at least 20% of each quarter square centimeter, e.g. 10 to 90%, in particular 20 to 80% of the area of each quarter square centimeter is not covered by any polymer coating.

The shape of area covered by polymer coating may be arbitrary as long as the above conditions with regard to the total coverage and the even distribution of the coverage are fulfilled. Preferably, the polymer coating is arranged on the surface of the sheet-like support such that a continuous, fully coated area is not greater than 22.5 mm², in particular not greater than 20 mm², especially not greater than 10 mm², e.g. in the range of 0.01 to 22.5 mm² or in the range of 0.02 to 20 mm² or in the range of 0.03 to 10 mm². In particular, the polymer coating is arranged on the surface of the sheet-like support as dots or dashes or combinations thereof. The dashes and the dots may have a regular shape, such as a circle or a straight line or may have an irregular form. Preferably, the size of the dots or dashes is not greater than 10 mm², e.g. in the range of 0.01 to 10 mm² or in the range of 0.02 to 5 mm² or in the range of 0.03 to 1 mm². The polymer coating may also be arranged on the surface of the sheet-like support as one or more grids. In this case, the distance between adjacent knots of the grid will usually not exceed 10 mm, in particular 5 mm and is typically in the range from 0.1 to 10 mm, especially in the range of 0.2 to 5 mm. The widths of the lines connecting the nots will usually be in the range of 0.1 to 3 mm. It is also possible that the polymer coating is arranged on the surface of the sheet-like support as a combination of at least one of dots or dashes and one or more grids, where dashes, dots and grids have the above dimensions. Especially, any polymer coating is arranged on the surface of the sheet-like support as dots or dashes or combinations thereof.

The polymer coating arranged on the surface of the carrier will usually have a thickness of at least 1 µm, in particular at least 2 µm and especially at least 3 µm, which corresponds to coating amounts applied to the sheet-like materials at 100% coverage of at least 1 g/m², in particular of at least 2 g/m², especially of at least 3 g/m². In particular, the total thickness of the polymer coating is in the range from 1 to 50 µm, in particular 2 to 40 µm, especially 3 to 30 µm, corresponding coating amounts applied to the sheet-like materials at 100% coverage of 1 to 50 g/m², in particular 2 to 40 g/m², especially 3 to 30 g/m².

The polymer coatings may essentially consist of one or more organic polymers but it may also contain other components conventionally present in polymer coatings, e.g. additives conventionally present in coating compositions, such as film forming additives, rheology modifying additives, emulsifiers, biocides, UV stabilizers and pH adjusting agents, pigments and fillers. Besides the organic polymer, the polymer coatings may also contain one or more waxes. In this context, the term “essentially consist of” means that the amount of organic polymer is at least 95% by weight, in particular at least 98% by weight of the polymer coating. Frequently, the amount of organic polymer in the polymer coating is at least 50% by weight, frequently at least 70% by weight, the remainder being selected from the group consisting of additives conventionally present in coating compositions, pigments, fillers and waxes and combinations thereof.

If the polymer coating contains one or more components selected from pigments and fillers, the total amount of pigment and fillers will usually not exceed the amount of polymer in the polymer coating. In particular, the weight ratio of the total amount of these components to the amount of polymer is < 1 and will usually not exceed a ratio of 1:1.1, in particular 1:1.5 and especially 1:2. If a component selected from pigments and fillers is present in the polymer coating, its amount is frequently in the range from 1 to 48% by weight, in particular in the range of 2 to 40% by weight or in the range of 3 to 30% by weight, based on the total weight of the polymer coating. In particular, the polymer coating may essentially consist of one or more organic polymers, the remainder, if any, being selected from the aforementioned conventional additives.

If the polymer coating contains one or more components selected from waxes, the total amount of waxes will usually not exceed the amount of polymer in the polymer coating. In particular, the weight ratio of waxes to the amount of polymer is < 1 and will usually not exceed a ratio of 1:1.1, in particular 1:1.5 and especially 1:2. If a component selected from waxes is present in the polymer coating, its amount is frequently in the range from 1 to 3% by weight, in particular in the range of 2 to 20% by weight or in the range of 3 to 15% by weight, based on the total weight of the polymer coating.

In particular, the polymer coating may essentially consist of one or more organic polymers or a combination of at least one organic polymer and at least one wax, the remainder, if any, being selected from the aforementioned conventional additives.

The polymer of the polymer coating may principally be any polymer or combination of polymers which have an evaporation retardant effect on odorant compounds, i.e. which significantly reduces the evaporation rate of a odorant compound when used as a continuous membrane. A significant reduction of the evaporation rate means that a polymer coating, which essentially consists of the polymer and which is coated to an 80 g/m² uncoated paper with a coverage of 100% of the surface and a coating thickness of 10 µm , will reduce the permeation rate at 23° C. and 1 bar by at least 50% compared to an uncoated paper.

Suitable polymers are for examples the following polymers: Acrylate polymers, including styrene acrylates, pure acrylates, ethylene-acrylate copolymers and acrylate rubbers (ACM), copolymers of alkyl acrylates with ethylene (AEM), polyester urethanes (AU), polybutadiene (BR), ethylene-acrylonitrile copolymers (ENM), ethylene-propylene-diene terpolymers (EPDM), ethylene-propylene copolymers (EPM), polyethylene (PE), polypropylene (PP), polyisobutene (PIB), polyether urethanes (EU), ethylene-vinyl acetate copolymers (EVM), fluorinated rubbers (FKM), fluorosilicone rubbers (FVMQ), isobutene-isoprene copolymers (IIR), isoprene rubbers (IR), nitrile rubbers (NBR), natural rubber (NR), thioplastics (OT), styrene-butadiene copolymers (SB), styrene-butadiene rubbers (SBR), carboxyl-containing acrylonitrile-butadiene copolymers (XNB), carboxyl-containing styrene-butadiene copolymers (XSB), carboxyl-containing styrene-butadiene rubbers (XSBR) and mixtures thereof.

In particular, the polymer of the polymer coating comprises or is at least one polymer selected from the group consisting of acrylate polymers, especially styrene acrylate polymers and pure acrylate polymers, styrene-butadiene rubbers (SBR), carboxyl-containing acrylonitrile-butadiene copolymers (XNB), carboxyl-containing styrene-butadiene copolymers (XSB), carboxyl-containing styrene-butadiene rubbers (XSBR) and mixtures thereof with particular preference to acrylate polymers.

In a particular group of embodiments, the polymer of the polymer coating is based on an aqueous polymer dispersion of one or more of the aforementioned polymers, in particular of an aqueous polymer dispersion of one or more polymer selected from the group consisting of acrylate polymers, especially styrene acrylate polymers and pure acrylate polymers, styrene-butadiene rubbers (SBR), carboxyl-containing acrylonitrile-butadiene copolymers (XNB), carboxyl-containing styrene-butadiene copolymers (XSB), carboxyl-containing styrene-butadiene rubbers (XSBR) and mixtures thereof with particular preference to aqueous polymer dispersions of acrylate polymers.

In a particular group of embodiments, the polymer is selected from the group consisting of styrene-butadiene copolymers, including SB, SBR, XSB and XSBR, and mixtures thereof. In this in particular group of embodiments, the polymer is in particular an aqueous dispersion of a styrene-butadiene copolymer. A styrene-butadiene copolymer is a polymer, which comprises polymerized repeating units of styrene and/or a styrene derivative and polymerized repeating units of butadiene. In such polymers, typically the polymerized butadiene is 1 ,2-linked and/or 1 ,4-linked, and the copolymers still have ethylenically unsaturated bonds which are susceptible to vulcanization. Preference is given to butadiene-styrene copolymers, in particular to their aqueous polymer dispersions, wherein the weight ratio of styrene to butadiene is in the range 10:90 to 90:10, in particular from 20:80 to 80:20, especially from 25:75 to 75:25. Usually, in these copolymers the total amount of polymerized styrene and butadiene is at least 80% by weight, in particular at least 85% by weight, especially at least 90% by weight, based on the total amount of monomers forming the styrene-butadiene copolymer. Besides styrene and butadiene, the styrene-butadiene copolymer may contain other monomers, i.e., include other monomer units. The amount of these other monomers will usually not exceed 20% by weight, in particular is 15% by weight or less, or 10% by weight or less, based on the total amount of monomers forming the styrene-butadiene copolymer.

In another particular groups of embodiments, the polymer is selected from the group consisting of acrylate polymers, including styrene acrylates, pure acrylates, ethylene-acrylate copolymers and acrylate rubbers, in particular selected from the group consisting of styrene acrylates, pure acrylates and mixtures thereof. In this in particular group of embodiments, the polymer is in particular an aqueous dispersion of an acrylate polymer, which is in particular selected from the group consisting of styrene acrylates, pure acrylates and mixtures thereof.

The term “styrene acrylates” as used in the art refers to copolymers of styrene with at least one acrylate ester and optionally one or more methacrylate esters. They are typically present in the form of aqueous polymer dispersions.

The term “pure acrylates” as used in the art refers to homo or copolymer of at least principal monomer M1 selected from methacrylate esters and acrylate esters, in particular copolymers of at least one monomer M 1a selected from methacrylate esters and at least one monomer M1b selected from acrylate esters. They are typically present in the form of aqueous polymer dispersions. The term “principal monomer” means that the total amount of monomer M1 is at least 50% by weight, frequently at least 80% by weight, in particular at least 85% by weight, especially at least 90% by weight, based on the total amount of monomers forming the pure acrylate.

Suitable acrylate esters in styrene acrylates and in pure acrylates are C₁-C₂₀ alkyl esters of acrylic acid and C₅-C₂₀-cyloalkyl esters of acrylic acid. Suitable methacrylate esters in styrene acrylates and pure acrylates are C₁-C₂₀ alkyl esters of methacrylic acid and C₅-C₂₀-cyloalkyl esters of methacrylic acid.

Examples of C₁-C₂₀ alkyl esters of acrylic acid, also termed C₁-C₂₀-alkyl acrylates, include, but are not limited to methyl acrylate, ethyl acrylate, n-propyl acrylate, isopropyl acrylate, n-butyl acrylate, 2-butyl acrylate, tert-butyl acrylate, n-pentyl acrylate, isopentyl acrylate, n-hexyl acrylate, n-heptyl acrylate, n-octyl acrylate, 2-ethylhexyl acrylate, 2-propylpentyl acrylate, n-decyl acrylate, 2-propylheptyl acrylate, C₁₀ isoamyl guerbet acrylate, 1-propylheptyl acrylate, lauryl acrylate and stearyl acrylate. Examples of C₁-C₂₀ alkyl esters of methacrylic acid, also termed C₁-C₂₀-alkyl methacrylates, include, but are not limited to methyl methacrylate, ethyl methacrylate, n-propyl methacrylate, isopropyl methacrylate, n-butyl methacrylate, 2-butyl methacrylate, tert-butyl methacrylate, n-pentyl methacrylate, isopentyl methacrylate, n-hexyl methacrylate, n-heptyl methacrylate, n-octyl methacrylate, 2-ethylhexyl methacrylate, 2-propylpentyl methacrylate, n-decyl methacrylate, 2-propylheptyl methacrylate, C₁₀ isoamyl guerbet methacrylate, 1-propylheptyl methacrylate, lauryl methacrylate and stearyl methacrylate. Examples C₅-C₂₀-cyloalkyl esters of acrylic acid include, but are not limited to cyclopentyl acrylate, cyclohexyl acrylate, 4-methylcyclohexyl acrylate and 4-tert-butylcyclohexyl acrylate. Examples of C₅-C₂₀-cyloalkyl esters of methacrylic include, but are not limited to cyclopentyl methacrylate, cyclohexyl methacrylate, 4-methylcyclohexyl methacrylate and 4-tert-butylcyclohexyl methacrylate.

In acrylate polymers, such as styrene acrylates and the pure acrylates, the total amount of styrene, acrylate esters and methacrylate esters is at least 80% by weight, in particular at least 85% by weight, especially at least 90% by weight, based on the total amount of monomers forming the styrene-butadiene copolymer. Besides styrene, acrylate esters and methacrylate esters, the acrylate polymers, such as styrene acrylates and the pure acrylates, may contain other monomers, i.e., include other monomer units. The amount of these other monomers will usually not exceed 20% by weight, in particular is 15% by weight or less, or 10% by weight or less, based on the total amount of monomers forming the respective copolymer.

The styrene-butadiene copolymers, the styrene acrylate copolymers and the pure acrylates can also include crosslinking monomers. The amount of crosslinking monomers will usually not exceed 5% by weight, in particular 2% by weight, based on the total amount of monomers forming the respective polymer. When used in the styrene-butadiene copolymers, styrene acrylate copolymers or pure acrylate polymers, the crosslinking monomers can e.g. be present in an amount of from 0.01 to 5% by weight or in an amount from 0.2 to 2% by weight, based on the total amount of monomers forming the respective polymer. Suitable crosslinking monomers are in particular monomers, which contain at least two non-conjugated ethylenically unsaturated double bonds. Exemplary crosslinking monomers include divinylbenzene, diesters or triesters of dihydric and trihydric alcohols with monoethylenically unsaturated C₃-C₆ monocarboxylic acids, e.g., di(meth)acrylates, tri(meth)acrylates), and tetra(meth)acrylates, e.g. alkylene glycol diacrylates and dimethacrylates, such as ethylene glycol diacrylate, 1 ,3-butylene glycol diacrylate, 1 ,4-butylene glycol diacrylate and propylene glycol diacrylate, trimethylolpropan triacrylate and trimethacrylate, pentaerythrit triacrylate and pentaerythrit tetraacrylate, but also vinyl and allyl esters of ethylenically unsaturated acids, such as vinyl methacrylate, vinyl acrylate, allyl methacrylate, allyl acrylate, and divinyl and diallyl esters of dicarboxyilic acids, such as diallyl maleate and diallyl fumarate and also methylenebisacrylamide. Suitable crosslinking monomers are having at least one ethylenically unsaturated double bond and a further reactive group susceptible to a post-crosslinking reaction, inlcuding ethylenically unsaturated monomers containing a keto group, e.g., acetoacetoxyethyl(meth)acrylate or diacetonacrylamide; monomers containing an urea group, e.g. ureidoethyl (meth)acrylate, silane crosslinkers, e.g. vinyl triethoxysilane, 3-methacryloxypropyl trimethoxysilane and 3-mercaptopropyl trimethoxy silane, epoxy functionalized (meth)acrylate monomers, e.g. glycidyl methacrylate, N-alkylolamides of α,β-monoethylenically unsaturated carboxylic acids having 3 to 10 carbon atoms and esters thereof with alcohols having 1 to 4 carbon atoms, e.g. N-methylolacrylamide and N-methylolmethacrylamide.

The styrene-butadiene copolymers, the styrene acrylate copolymers and the pure acrylate polymers may include repeating units of acid monomers. The amount of these acid monomers will usually not exceed 10% by weight, in particular is 5% by weight or less, based on the total amount of monomers forming the respective polymer. When used in the styrene-butadiene copolymers, styrene acrylate copolymers or pure acrylate polymers, the acid monomers can e.g. be present in an amount of from 0.01 to 10% by weight or in an amount from 0.1 to 5% by weight, based on the total amount of monomers forming the respective polymer. Acid monomers have usually one acid group, such as a carboxyl group (COOH), sulfonic acid groups (SO₃H), phosphonic acid groups and phosphate groups. Examples acid monomers are monoethylenically unsaturated C₃-C₆-monocarboxylic acids, such as acrylic acid, methacrylic acid, and monoethylenically unsaturated C₄-C₆-dicarboxylic acids, such as itaconic acid and fumaric acid and mixtures thereof. Examples acid monomers are also monoethylenically unsaturated sulfonic acids, such as vinyl sulfonic acid, styrene sulfonic acid, acryloxyethansulfonic acid and acrylamido-2-methylpropane sulfonic acid and the salts thereof, in particular the alkalimetal salts thereof, also monoethylenically unsaturated sulfonic acids.

Further monomers of styrene-butadiene copolymers may be non-ionic ethylenically unsaturated monomers, for example, conjugated diene monomers different from butadiene, e.g., isoprene or chloroprene, vinyl aromatic monomers, such as α-methylstyrene or o-chlorostyrene, ethylenecially unsaturated nitriles, such as acrylonitrile or methacrylonitrile, amides of ethylenecially unsaturated acids, such as acrylamide or methacrylamide, and C₁-C₁₀-alkyl esters of acrylic acid or methacrylic acid, such as methyl, ethyl, n-butyl, isobutyl and 2-ethylhexyl acrylates and methyl, ethyl, n-butyl, isobutyl and 2-ethylhexyl methacrylates. The total amount of these non-ionic monomers will usually not exceed not exceed 10% by weight, in particular is 5% by weight or less, based on the total amount of monomers forming the respective polymer.

Further monomers of styrene acrylate copolymers and the pure acrylate polymers may be non-ionic ethylenically unsaturated monomers having an increased solubility in deionized water of e.g. at least 80 g/L at 20° C. and 1 bar, for example, ethylenecially unsaturated nitriles, such as acrylonitrile or methacrylonitrile, amides of ethylenecially unsaturated acids, such as acrylamide or methacrylamide, and C₂-C₄-hydroxyalkyl esters of acrylic acid or methacrylic acid, such 2-hydroxyethyl acrylate, 2-hydroxyethyl methacrylate, hydroxypropyl acrylate and hydroxylpropyl methacrylate, monoethylenically unsaturated monomers bearing a keto group, such as diacetone acrylamide and diacetone methacrylamide and monoethylenically unsaturated monomers bearing an urea group, such as 2-(2-oxo-imidazolidin-1-yl)ethyl (meth)acrylate, 2-ureido (meth)acrylate, N-[2-(2-oxooxazolidin-3-yl)ethyl] methacrylate. The total amount of these non-ionic monomers will usually not exceed 10% by weight, in particular is 5% by weight or less, based on the total amount of monomers forming the respective polymer.

In a particularly preferred group of embodiments, the polymer is an acrylate polymer, in particular a styrene acrylate copolymer or a pure acrylate polymer, which has been prepared in the presence of at least one carbohydrate compound. In these polymers, the carbohydrate forms part of the polymer. In these carbohydrate containing polymers, the relative amount of polymerized monomers given for the acrylate polymers only refer to the total amount of ethylenically unsaturated monomers.

The carbohydrate compound may be selected from oligosaccharides (constructed of 2 to 10 saccharide units) and polysaccharides (constructed of more than 10 saccharide units), especially from degraded polysaccharides, preferably degraded starch, degraded hemicelluloses or degraded chitosan. Maltodextrin and glucose syrup are particularly preferred. The carbohydrate compound is preferably present in an amount from 10 to 200 parts by weight and more preferably from 20 to 150 parts by weight or from 30 to 150 parts by weight of carbohydrate compound per 100 parts by weight of ethylenically unsaturated monomer to be polymerized. The carbohydrate compounds, more particularly the degraded starches, have for example an intrinsic viscosity ηi of less than 0.07 dl/g or less than 0.05 dl/g. The intrinsic viscosity ηi is preferably in the range from 0.02 to 0.06 dl/g. The intrinsic viscosity ηi is determined in accordance with DIN EN 1628 at a temperature of 23° C.

The DE value is an alternative way to characterize the degree of degradation of polysaccharides, more particularly of starches which is very common in the field. DE denotes Dextrose Equivalent and refers to the percentage fraction of reducing sugar in the dry substance. It corresponds to the amount of glucose (= dextrose) which would have the same reducing power per 100 g of dry substance. The DE value is a measure of how far polymer degradation has proceeded; hence products obtained having a low DE value retain a high proportion of polysaccharides and a low content of low molecular weight sugars, while products of high DE value are mainly made up of just low molecular weight sugars only. Examples of suitable degraded starches are maltodextrin and glucose syrup. Preferred maltodextrins have intrinsic viscosities in the range of not less than about 0.04 to 0.06 dl/g, DE values of 3 to 20 and molar masses Mw in the range from 15,000 to 20,000 g/mol.

Usually, the polymer of the polymer coating is thermoplastic. Typically, the polymer of the polymer coating has a softening or melting point in the range from -30° C. to +60° C., preferably -20° C. to +50° C., especially -10° C. to +40° C., as determined by the differential scanning calorimetry (DSC). Frequently, the polymer of the polymer coating is characterized by having at least one glass transition temperature T_{G} in the range from -30° C. to +60° C., preferably -20° C. to +50° C., especially -10° C. to +40° C., as determined by the differential scanning calorimetry (DSC) method according to ISO 11357-2:2013, preferably with sample preparation according to ISO 16805:2003.

Typical waxes, which may be used in combination with the polymer of the polymer coating include paraffinic waxes, Montan waxes, including chemically modified Montan waxes and Montan ester waxes, oxidized waxes, amide waxes, polar polyolefin waxes, Fischer-Tropsch waxes, oxidation products of Fischer-Tropsch waxes and Sasol waxes. If the polymer of the polymer coating is present as an aqueous polymer dispersion it is preferred to use a combination of such an aqueous polymer dispersion with an aqueous dispersion of a wax.

If the respective polymer is present in the form of an aqueous polymer dispersion, the polymer is present in form of particles, which are dispersed in an aqueous serum. Frequently, the dispersed polymer particles of the aqueous polymer dispersions have a volume average particle diameter, also termed as D(4.3) value, in the range from 30 nm to 1 µm. In particular, the particle size may range from 50 nm to 0.8 µm or from 60 nm to 0.5 µm. The values given here refer to the values as determined by quasielastic light scattering (QELS), also known as dynamic light scattering (DLS). The measurement method is described in the ISO 13321:1996 standard.

If the respective polymer is present in the form of an aqueous polymer dispersion, the aqueous polymer dispersion has preferably a solids content of at least 10% by weight, in particular at least 20% by weight, preferably in the range from 10 to 72% by weight, especially 20 to 70% by weight, based on the total weight of the aqueous polymer dispersion. If polymer dispersions are used, which have a higher solids content, it may be necessary to dilute them with water prior to preparing a coating therefrom. The solids content is the amount of non-volatile matter in the aqueous polymer dispersion as determined in accordance with DIN EN ISO 3251 :2008-06.

In addition to the polymer, the aqueous polymer dispersion typically contains at least one surface active compound. The surface active compound serves to stabilize the aqueous dispersion of the polymer by keeping the particles of the polymer dispersed. The surface active compound may be an emulsifier, a protective colloid or a mixture of both of them. The emulsifier and the protective colloid are distinct from each other by their weight-average molar mass M_{w}. An emulsifier has typically a weight-average molar mass M_{w} in general below 2000, while the weight-average molar mass M_{w} of the protective colloid may be up to 50 000, in particular from above 2000 to up to 50 000. Typically, the amount of the surface active compound is in the range from 0.1 to 10% by weight, in partiuclar in the range from 0.5 to 5% by weight, based on the total amount of polymer in the aqueous polymer dispersion. In case of protective colloids, the amount of protective colloid may however be higher, e.g. up to 20% by weight, based on the total amount of dispersed polymer and protective colloid in the aqueous dispersion.

Preferably, the surface active compound comprises one or more emulsifiers. The emulsifier is non-ionic, anionic, or cationic. In case of employing a mixture of emulsifiers, their compatibility has to be assured, which can be evaluated in case of doubt by preliminary tests. Typically, an anionic emulsifier is compatible with another anionic emulsifier or a non-ionic emulsifier. Similarly, a cationic emulsifier is typically compatible with another cationic emulsifier or a non-ionic emulsifier. Preferably, the emulsifier is an anionic emulsifier, a combination of two or more anionic emulsifiers or a combination of at least one anionic emulsifier and at least one non-ionic emulsifier.

Non-ionic emulsifier are, for example, ethoxylated C₈-C₃₆ fatty alcohols having a degree of ethoxylation of from 3 to 50 (= ethylene oxide units [EO]: 3-50) and ethoxylated mono-, di- and tri-C₄-C₁₂ alkylphenols having a degree of ethoxylation of from 3 to 50. Examples of customary nonionic emulsifiers are the Emulgin B grades (cetyl/stearyl alcohol ethoxylates, RTM BASF), Dehydrol LS grades (fatty alcohol ethoxylates, EO units: 1-10, RTM BASF), Lutensol A grades (C₁₂ C₁₄-fatty alcohol ethoxylates, EO units: 3-8, RTM BASF), Lutensol AO grades (C₁₃ C₁₅-_{OXO} alcohol ethoxylates, EO units: 3-30), Lutensol AT grades (C₁₆ C₁₈-fatty alcohol ethoxylates, EO units: 11-80), Lutensol ON grades (C₁₀-oxo alcohol ethoxylates, EO units: 3-11) and Lutensol TO grades (C₁₃-_{OXO} alcohol ethoxylates, EO units: 3-20). Here and in the following the phrase “EO units” means the number average of ethylene oxide repeating units in the emulsifier.

Anionic emulsifiers are for example the alkali metal salts of dialkyl esters of sulfosuccinic acid, the alkali metal salts and the ammonium salt of C₈-C₁₂ alkyl sulfates, the alkali metal salts and the ammonium salts of C₁₂-C₁₈ alkylsulfonic acids, the alkali metal salts and the ammonium salts of C₉-C₁₈ alkylarylsulfonic acid, the alkali metal salts and the ammonium salts of sulfuric acid monoesters of ethoxylated C₁₂-C₁₈ alkanols (EO units: 4-30) or a sulfuric acid monoester of an ethoxylated (C₄-C₁₂ alkyl)phenol (EO units: 3-50). As further anionic emulsifiers, compounds of the general formula I

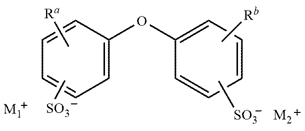

wherein R^{a} and R^{b} are each a H atom or C₄-C₂₄-alkyl and are not both H atoms at the same time, and M₁⁺ and M₂⁺ can be alkali metal ions and/or ammonium, are also useful. In the general formula I, R^{a} and R^{b} are preferably linear or branched alkyl radicals having from 6 to 18 carbon atoms, in particular 6, 12 or 16 carbon atoms, or hydrogen atoms, where R^{a} and R^{b} are not both hydrogen atoms at the same time. M₁⁺ and M₂⁺ are preferably sodium, potassium or ammonium, with sodium being particularly preferred. A compound of general formula I, in which M₁⁺ and M₂⁺ are both sodium, R^{a} is a branched alkyl radical having 12 carbon atoms and R^{b} is hydrogen or R^{a} is particularly advantageous. Use is frequently made of industrial mixtures which have a proportion of from 50 to 90% by weight of the monoalkylated product, for example Dowfax® 2A1 (RTM The Dow Chemical Corp.). The compounds of general formula I are commonly known, e.g. from US-A 4 269 749, and commercially available. Further anionic emulsifiers are fatty alcohol phosphates, alkylphenol phosphates, alkyl polyglycol ether phosphates, alkyl polyalkylene oxide phosphates, and fatty alcohol ether phosphates and the salts thereof, in particular the alkalimetal salts and ammonium salts thereof, with particular preference given to the alkalimetal salts, such as sodium salts.

A comprehensive description of suitable emulsifiers may be found in Houben-Weyl, Methoden der organischen Chemie, volume XIV/1, Makromolekulare Stoffe, Georg-Thieme-Verlag, Stuttgart, 1961, pages 192 to 208.

Like the aforementioned emulsifiers, suitable protective colloids may be non-ionic, anionic or cationic. Examples of protective colloids are poly(vinyl alcohols), poly(alkylene glycols), poly(acrylic acids) and the alkali metal salt thereof, poly(methacrylic acids) and the alkali metal salt thereof, and gelatin derivatives. Anionic protective colloids can also be a copolymer, containing a suitable amount of at least one anionic monomer, such as acrylic acid, methacrylic acid, maleic acid, 2-acrylamido-2-methylpropane sulfonic acid, para-vinylphenyl sulfonic acid or salt forms thereof, preferably alkali metal salts thereof, in polymerized form. Examples of cationic protective colloids are homopolymers and copolymers containing a sufficient amount of cationic monomers, in particular monoethylenically unsaturated monomers having one or more amino groups, which are N-protonated or N-alkylated. Examples include N-protonated and N-alkylated derivatives of homopolymers or copolymers of N-vinylformamide in their at least partly hydrolyzed form, homopolymers or copolymers of N-vinylacetamide in their at least partly hydrolyzed form, N-protonated and N-alkylated derivatives of homopolymers or copolymers of N-vinylcarbazole, N-protonated and N-alkylated derivatives of homopolymers or copolymers of 1-vinylimidazole, N-protonated and N-alkylated derivatives of homopolymers or copolymers of 2-vinylimidazole, N-protonated and N-alkylated derivatives of homopolymers or copolymers of 2-vinylpyridine, N-protonated and N-alkylated derivatives of homopolymers or copolymers of 4-vinylpyridine, N-protonated and N-alkylated derivatives of homopolymers or copolymers of amine-group-bearing acrylates, N-protonated and N-alkylated derivatives of homopolymers or copolymers of amine-group-bearing methacrylates, wherein the nitrogen of the amine-group is protonated at a pH below 7 or is permanently positively charged, for example by alkylation. Further comonomers in such cationic protective colloids may be acrylamide, methacrylamide and N-vinyl pyrrolidone.

The protective colloids are distinct from the polymers dispersed in the aqueous polymer dispersion, as they are water-soluble or water dispersible. The term “water-soluble or water dispersible” is understood that the corresponding protective colloid can be dissolved or dispersed in deionized water at 20° C. and 1013 mbar in an amount of at least 10 g/L polymer such that the resulting aqueous solution has either no measurable particle size or a particle size of at most 20 nm as determined by dynamic light scattering in accordance with DIN 22412:2008.

A comprehensive description of suitable protective colloids may be found in Houben-Weyl, Methoden der organischen Chemie, volume XIV/1, Makromolekulare Stoffe, Georg-Thieme-Verlag, Stuttgart, 1961, pages 411 to 420.

The aqueous polymer dispersions, in particular the aqueous polymer dispersions of styrene-butadiene copolymers and acrylate polymers, are well known to a skilled person and usually prepared by a radical aqueous emulsion polymerization technique, such as described in “Emulsionspolymerisation” [Emulsion Polymerization] in Encyclopedia of Polymer Science and Engineering, vol. 8, pages 659 ff. (1987); D. C. Blackley, in High Polymer Latices, vol. 1, pages 35 ff. (1966); H. Warson, The Applications of Synthetic Resin Emulsions, chapter 5, pages 246 ff. (1972); D. Diederich, Chemie in unserer Zeit 24, pages 135 to 142 (1990); Emulsion Polymerisation, Interscience Publishers, New York (1965); Dispersionen synthetischer Hochpolymerer [Dispersions of Synthetic High Polymers], F. Hölscher, Springer-Verlag, Berlin (1969)], WO 2012/049651, WO 2012/163749, WO 2012/163821 and WO 2013/083504, which is hereby incorporated by reference.

As mentioned before, the coating may contain a component, which is selected from fillers and pigments. Pigments and fillers for use in coating compositions are well known to a skilled person. White pigments/fillers are contemplated in particular. Suitable pigments and fillers include, for example, metal salt pigments/fillers, such as, for example, calcium sulfate, calcium aluminate sulfate, barium sulfate, magnesium carbonate and calcium carbonate. The calcium carbonate may be natural ground calcium carbonate (GCC), precipitated calcium carbonate (PCC), lime or chalk. Suitable calcium carbonate pigments are available for example as Covercarb® 60, Hydrocarb® 60 or Hydrocarb® 90 ME. Further suitable pigments/fillers include, for example, silicas, aluminas, aluminum hydrate, silicates, titanium dioxide, zinc oxide, kaolin, argillaceous earths, talc or silicon dioxide. Suitable further pigments/fillers are available for example as Capim® MP 50 (Clay), Hydragloss® 90 (Clay) or Talcum C10. In one embodiment of the invention, the polymer coating comprises a combination of at least one polymer and for example up to 1 part by weight of pigments/fillers, preferably platelet-shaped pigments/filler, based on 1 part by weight of polymer. Examples of platelet-shaped pigments are talc, clay or mica (glimmer). In this embodiment, talc is a preferred platelet pigment/filler. Preferred aspect ratios (ratio of length to thickness) platelet pigment/fillers are above 10.

According to the invention, the polymer coating is arranged on the surface of a sheet-like support, which is permeable to odorants. Suitable carriers are typically porous materials, where the pores allow for permeation of liquid odorant compositions by capillary action. Preferably, the carrier is paper or cardboard according to the definition, in particular a paper having a paper grammage according to DIN EN ISO 536:2019-04 in the range of 20 to 200 g/m². In particular, the paper is uncoated.

The evaporation retardant membranes of the invention may have a single polymer coating arranged on one surface of the sheet-like support or two or more polymer coatings, which may be arranged on the same surface of the sheet-like support. Alternatively, one or more polymer coatings are arranged on one surface and one or more further polymer coatings are arranged on the other (opposite) surface of the sheet-like support.

In a particular group of embodiments, the evaporation retardant membrane comprises at least two different polymer coatings. In this group of embodiments, the polymer coatings are preferably arranged on one surface of the sheet-like support such that the coverage of the surface by the respective polymer coating is 5 to 85%, in particular 10 to 70%. The area of the surface covered by one of the at least two different coatings will usually be at least 1%, in particular at least 10%, especially at least 20% of the surface area covered by all coatings and may be from 1 to 99%, in particular from 10 to 90% and especially from 20 to 80% of the surface area covered by all coatings. In case of two coatings, the ratio of the areas covered by the respective coatings is in the range from 1:99 to 99:1, in particular in the range of 10:90 to 90:10 and especially in the range from 20:80 to 80:20. In this group of embodiments, the total coverage of the surface by all polymer coatings, which are present on said surface, is usually 10 to 90%, in particular 20 to 80% of the total area of the surface, which forms the evaporation retardant membrane. Consequently, 10 to 90%, in particular 20 to 80% of the total area of said surface is not covered by any polymer coating.

Preferably, the different polymer coatings are arranged on different areas of one of the surfaces of the sheet-like support. The areas of said surface covered by the same polymer coating may be quite small of less than 1 mm² or in the range from 1 mm² to at most 99% of the total area covered by all polymer coatings on a surface, in particular from 2 mm² to at most 90% of the total area covered by all polymer coatings, especially from 3 mm² to at most 80% of the total area covered by all polymer coatings. The area, which is covered by the same polymer coating may be continuous are or 2 or more different areas.

The different polymer coatings may be distinct from each other by principally any feature, which might affect the diffusion rate of a odorant compound, including thickness of the polymer coating, coverage, polarity of the polymer contained in the coating, molecular constitution of the polymer in the polymer coating, in particular its glass transition temperature and its polarity, and the relative amount of polymer in the polymer coating. In particular, the different polymer coatings are distinct from each other by polarity and/or by their glass transition temperature of the polymer contained therein.

The glass transition temperature and the polarity of the polymer depend on the molecular architecture of the polymer, in particular by the kind of repeating units in a well known manner.

For example, the glass transition temperature of a copolymer can be calculated from the glass transition temperature by the so-called Fox equation (1)

$${\text{1/Tg}}^{\text{t}} {\text{=x}}_{\text{a}} {\text{/Tg}}_{\text{a}} {\text{+x}}_{\text{b}} {\text{/Tg}}_{\text{b}} \text{+} .... {\text{x}}_{\text{n}} {\text{/Tg}}_{\text{n}} ,$$

In this equation xₐ, x_{b,} ........ _{Xn} are the mass fractions of the monomers a, b, .... .... n and Tgₐ, T_{gb}, .... .... Tgₙ are the actual glass transition temperatures in Kelvin of the homopolymers synthesized from only one of the monomers 1, 2, ........ n at a time. The Fox equation is described by T. G. Fox in Bull. Am. Phys. Soc. 1956, 1, page 123 and as well as in Ullmann’s Encyclopadie der technischen Chemie [Ullmann’s Encyclopedia of Industrial Chemistry], vol. 19, p. 18, 4th ed., Verlag Chemie, Weinheim, 1980. The actual Tg values for the homopolymers of most monomers are known and listed, for example, in Ullmann’s Encyclopadie der technischen Chemie [Ullmann’s Encyclopedia of Industrial Chemistry], 5th ed., vol. A21, p. 169, Verlag Chemie, Weinheim, 1992. Further sources of glass transition temperatures of homopolymers are, for example, J. Brandrup, E. H. Immergut, Polymer Handbook, 1st Ed., J. Wiley, New York 1966, 2nd Ed. J. Wiley, New York 1975, 3rd Ed. J. Wiley, New York 1989 and 4th Ed. J. Wiley, New York 2004.

For example, the monomer composition forming the polymer of one of the polymer coatings is chosen such that its theoretical glass transition temperature Tg^{t} is in the range of -30 to+20° C., in particular in the range of -20° C. to +15° C., especially in the range of -10° C. to +10° C., while the monomer composition forming the polymer of the other polymer coating is chosen such that its theoretical glass transition temperature Tg^{t} is in the range of +5 to+60° C., in particular in the range of +10° C. to +50° C., especially in the range of +15° C. to 40° C., provided that the difference of the glass transition temperature is at least 5° C., in particular at least 10° C., especially at least 15° C.

Likewise, polarity of the polymer depend on the monomer composition, which forms the polymer. The polarity of the polymer and the monomer correspond to their hydrophobicity, which can be estimated by the so called Hansch parameters HP, which are generally a measure of the hydrophobicity of monomers and the polymers formed therefrom. Here, a high Hansch parameter HP indicates a high hydrophobicity and a low polarity.

The theoretical considerations for the calculation of the Hansch parameters are disclosed in: Hansch, Fujita, J. Amer. Chem. Soc., 1964, 86, pages 1616-1626; H. Ku-binyi, Methods and Principles of Medicinal Chemistry, Volume 1, R. Mannhold et al., publisher: VCH, Weinheim (1993); C. Hansch and A. Leo, Substituent Constants for Correlation Analysis, in Chemistry and Biology, Wiley, New York (1979); and C. Hansch, P. Maloney, T. Fujita, and R. Muir, Nature, 1962, 194, pages 178-180.

In the context of the present document, the Hansch parameters for the monomers are generally calculated with the “KOWWIN v1.68” (September 2010) software which is made available to the public by the US Environmental Protection Agency (EPA) as “Estimation Programs Interface Suite™ for Microsoft® Windows, v4.11” [2012], United States Environmental Protection Agency, Washington, DC, USA. This program ascertained the Hansch parameters for the monomers that were among those used in this document. If the polymers are used in the form of their aqueous polymer dispersions, which advantageously have a pH in the neutral to slightly alkaline range, complete deprotonation is assumed for the monomers containing acid groups, and so the calculation is made with the salt specified in each case. The following table 1 gives a list of Hansch parameters of monomers, which are frequently used for producing polymers suitable as polymers for polymer coatings according to the present invention:

Table 1

Hansch parameters HP of monomers

| Monomer | HP |
|---|---|
| Phosphoethyl methacrylate (as disodium salt, estimation) | -5.6 |
| Itaconic acid (as disodium itaconate) | -5.6 |
| Maleic acid (as disodium maleate) | -5.21 |
| Vinylsulfonic acid (as sodium vinylsulfonate) | -4.17 |
| Acrylic acid (as ammonium acrylate) | -2.43 |
| Methacrylic acid (as ammonium methacrylate) | -1.89 |
| Vinyltrimethoxysilane | -0.31 |
| Hydroxyethyl acrylate | -0.25 |
| Acrylonitrile | 0.21 |
| Acetoacetoxyethyl methacrylate | 0.24 |
| Tetraethylene glycol diacrylate | 0.29 |
| Hydroxyethyl methacrylate | 0.3 |
| Ureidomethacrylate | 0.41 |
| Vinyl acetate | 0.73 |
| Methyl acrylate | 0.73 |
| 3-Methacryloyloxypropyltrimethoxysilane | 0.75 |
| Vinyltriethoxysilane | 1.16 |
| Ethyl acrylate | 1.22 |
| Methyl methacrylate | 1.28 |
| Butanediol diacrylate | 2.1 |
| Allyl methacrylate | 2.12 |
| Isobutyl acrylate | 2.13 |
| n-Butyl acrylate | 2.2 |
| Isobutyl methacrylate | 2.67 |
| n-Butyl methacrylate | 2.75 |
| Styrene | 2.89 |
| Hexanediol diacrylate | 3.08 |
| Alpha-Methylstyrene | 3.44 |
| 2-Ethylhexyl acrylate | 4.09 |
| 2-Ethylhexyl methacrylate | 4.64 |
| Isobornyl methacrylate | 4.76 |
| Lauryl acrylate | 6.13 |
| Lauryl methacrylate | 6.68 |
| Stearyl acrylate | 9.62 |

The Hansch HP parameters can generally be calculated for a polymer P formed from monomers M1, M2 ... Mn by the following general formula (2):

$${\text{HP}}_{\text{P}} {\text{=x}}_{\text{1}} ⋅ {\text{HP}}_{\text{M1}} {\text{+x}}_{\text{2}} ⋅ {\text{HP}}_{\text{M2}} + .... {\text{x}}_{\text{n}} . {\text{HP}}_{\text{Mn}}$$

with
HPₚ: calculated Hansch parameter of the polymer P formed from the monomers M1, M2 ... Mn
x₁, x₂, xₙ: proportions by weight of the monomers M1, M2 .... Mn incorporated into the polymer P in percent divided by 100, where the sum total of x₁ + x₂ + ..... xₙ = 1
HP_{M1}, HP_{M2}, HP_{Mn}: the individual Hansch parameters of each of the monomers M1, M2 .... Mn which form the polymer P.

Typical polymers used in the polymer coatings of the present invention will have Hansch parameters in the range of 0.5 to 4.5, in particular in the range of 1.0 to 4.2. For example, the monomer composition forming the polymer of one of the polymer coatings is chosen such that its Hansch parameter is in the range of 0.5 to 3.0, especially in the range of 1.0 to 2.5, while the monomer composition forming the polymer of the other polymer coating is chosen such that its Hansch parameter is in the range of 2.0 to 4.5, especially in the range of 2.5 to 4.0, provided that the difference of Hansch parameters is at least 0.1, in particular at least 0.2, especially at least 0.5.

The polymer coating can be obtained by applying one or more polymer compositions containing the respective polymers to the surface of the sheet-like support material in a manner that the desired degree coverage with the respective polymer coating is achieved. Preferably, the one or more polymer compositions will be applied by a printing process, including offset-printing processes, rotogravure printing processes, silk-screen printing processes, copperplate intaglio printing processes, flexographic printing processes, letterpress printing processes. The aforementioned printing techniques are well known to a skilled person.

For most application purposes, it has been shown beneficial, when the polymer coating is obtainable by applying an aqueous polymer composition containing a polymer binder in the form of an aqueous polymer dispersion to the surface of the sheet-like support. In particular, the aqueous polymer composition contains an aqueous polymer dispersion of a polymer selected from the group consisting of acrylate polymers, especially styrene acrylate polymers and pure acrylate polymers, styrene-butadiene rubbers (SBR), carboxyl-containing acrylonitrile-butadiene copolymers (XNB), carboxyl-containing styrene-butadiene copolymers (XSB), carboxyl-containing styrene-butadiene rubbers (XSBR) and mixtures thereof. These aqueous polymer compositions are particularly beneficial, if the polymer coating is applied to the surface of the sheet-like support by a printing process.

Besides the one or more organic polymers and water, the aqueous coating composition may also contain other components conventionally present in polymer coatings, e.g. additives conventionally present in coating compositions, such as film forming additives, rheology modifying additives, emulsifiers, biocides, UV stabilizers and pH adjusting agents, and also pigments and fillers. Frequently, the amount of organic polymer in the aqueous coating composition is at least 50% by weight, frequently at least 70% by weight, based on the total amount of non-aqueous ingredients of the aqueous coating composition, the remainder being additives conventionally present in coating compositions, pigments and fillers. In particular, the weight ratio of the total amount of these components to the amount of polymer is < 1 and will usually not exceed a ratio of 1:1.1, in particular 1:1.5 and especially 1:2. If a component selected from pigments and fillers is present in the aqueous coating composition, its amount is frequently in the range from 1 to 48% by weight, in particular in the range of 2 to 40% by weight or in the range of 3 to 30% by weight, based on the total amount of the non-aqueous ingredients of the aqueous coating composition. The solids content of the aqueous coating compositions is typically in the range of 20 to 80% by weight.

The evaporation retardant membranes of the invention allows for controlled release of odorant compositions. These odorant compositions contain one or more odorant compounds, in particular at least two odorant compounds, more particularly three or more odorant compounds.

In the context of the present invention, “odorant” is understood to mean natural or synthetic substances having intrinsic odor. In the context of the present invention, “odor” or “olfactory perception” is the interpretation of the sensory stimuli, which are sent from the chemoreceptors in the nose or other olfactory organs to the brain of a living being. The odor can be a result of sensory perception of the nose of odorant, which occurs during inhalation. In this case, the air serves as odor carrier.

Typically, the odorant compositions are liquid at 22° C. and 1013 mbar. Typically, the odorant compounds contained in the composition are volatile organic compounds, which have a low polarity and, especially at 25° C., have a water solubility in deionized water of not more than 100 mg/L.

Volatile odorants are understood to mean odorants having a high vapor pressure at room temperature. A compound is considered to be volatile especially when it has the following property: If a droplet of the volatile fragrance is applied to a strip of paper and left to evaporate off under ambient conditions at room temperature (22° C.), its odor is no longer perceptible to an experienced perfumer no longer than 2 hours after application. Typically, odorant compounds at 25° C. have a measurable vapor pressure, which is typically at least 0.1 Pa and may be as high as 2 × 10⁴ Pa.

Hydrophobicity/polarity of the olfactory compounds is typically assessed by the partition coefficient, the compound in a mixture of n-octanol and water, P_{ow}. It is typically given as its decadal logarithm log Pow. Typically, an odorant has a log Pow of at least 0.5, e.g. in the range of 0.5 to 5.

The evaporation retardant membranes of the present invention are particularly suitable for the release of odorant compositions which comprise at least two different fragrance compounds, more particularly three or more odorant compounds, where the different odorant compounds have different polarities and/or different vapor pressures. A different vapor pressure means that ratio of the vapor pressures of the different odorant compounds is at least 1.5, in particular at least 2. A different polarity means that the differences between the log Pow of the compounds is at least 0.05, especially at least 0.1.

Odorant compounds are, for example, from the following compounds:

alpha-hexylcinnamaldehyde, 2-phenoxyethyl isobutyrate (Phenirat¹), dihydromyrcenol (2,6-dimethyl-7-octen-2-ol), methyl dihydrojasmonate (preferably having a cis isomer content of more than 60% by weight) (Hedione⁹, Hedione HC⁹), 4,6,6,7,8,8-hexamethyl-1,3,4,6,7,8-hexahydrocyclopenta[g]benzopyran (Galaxolide³), tetrahydrolinalool (3,7-dimethyloctan-3-ol), ethyl linalool, benzyl salicylate, 2-methyl-3-(4-tert-butylphenyl)propanal (Lilial²), cinnamyl alcohol, 4,7-methano-3a,4,5,6,7,7a-hexahydro-5-indenyl acetate and/or 4,7-methano-3a,4,5,6,7,7a-hexahydro-6-indenyl acetate (Herbaflorat¹), citronellol, citronellyl acetate, tetrahydrogeraniol, vanillin, linalyl acetate, styrenyl acetate (1-phenylethyl acetate), octahydro-2,3,8,8-tetramethyl-2-acetonaph-thone and/or 2-acetyl-1,2,3,4,6,7,8-octahydro-2,3,8,8-tetramethylnaphthalene (Iso E Super³), hexyl salicylate, 4-tert-butylcyclohexyl acetate (Oryclone¹), 2-tert-butylcyclohexyl acetate (Agrumex HC¹), alpha-ionone (4-(2,2,6-trimethyl-2-cyclohexen-1-yl)-3-buten-2-one), n-alpha-methylionone, alpha-isomethylionone, coumarin, terpinyl acetate, 2-phenylethyl alcohol, 4-(4-hydroxy-4-methylpentyl)-3-cyclohexenecarboxaldehyde (Lyral³), alpha-amylcinnamaldehyde, ethylene brassylate, (E)- and/or (Z)-3-methylcyclopentadec-5-enone (Muscenone⁹), 15-pentadec-11-enolide and/or 15-pentadec-12-enolide (Globalide¹), 15-cyclopentadecanolide (Macrolide¹), 1-(5,6,7,8-tetrahydro-3,5,5,6,8,8-hexamethyl-2-naphthalenyl)ethanone (Tonalide¹⁰), 2-isobutyl-4-methyltetrahydro-2H-pyran-4-ol (Florol⁹), 2-ethyl-4-(2,2,3-trimethyl-3-cyclo-penten-1-yl)-2-buten-1-ol (Sandolene¹), cis-3-hexenyl acetate, trans-3-hexenyl acetate, trans-2-cis-6-nonadienol, 2,4-dimethyl-3-cyclohexenecarboxaldehyde (Vertocitral¹), 2,4,4,7-tetramethyloct-6-en-3-one (Claritone¹), 2,6-dimethyl-5-hepten-1-al (Melonal²), borneol, 3-(3-isopropylphenyl)butanal (Florhydral²), 2-methyl-3-(3,4-methylenedioxyphenyl)propanal (Helional³), 3-(4-ethylphenyl)-2,2-dimethylpropanal (Florazon¹), tetra-hydro-2-isobutyl-4-methyl-2H-pyran (Dihydrorosenon⁴), 1,4-bis(ethoxymethyl)cyclohexane (Vertofruct⁴), L-isopulegol (1R,2S,5R)-2-isopropenyl-5-methylcyclohexanol, pyranyl acetate (2-isobutyl-4-methyltetrahydropyran-4-yl acetate), nerol ((Z)-2,6-dimethyl-2,6-octadien-8-ol), neryl acetate, 7-methyl-2H-1,5-benzo-dioxepin-3(4H)-one (Calone¹⁹⁵¹⁵), 3,3,5-trimethylcyclohexyl acetate (preferably with a content of cis isomers of 70% by weight) or more and 2,5,5-trimethyl-1 ,2,3,4,4a,5,6,7-octahydronaphthalen-2-ol (Ambrinol S¹), tetrahydro-4-methyl-2-(2-methylpropenyl)-2H-pyran (rose oxide), 4-methyl-2-(2-methylpropyl)oxane or 4-methyl-2-(2-methylpropyl)-2H-pyran (Dihydrorosan⁴), 2-isobutyl-4-methyltetrahydropyran-4-yl acetate, 2,2-dimethylpropane-1,3-diol diacetate (Velberry⁴), mixture of α- and β-santalol (Isobionics® Santalol), esters as disclosed in WO 2020/016421, preferably esters according to the mixture of example 1.1 (Florascone⁴), prenyl acetate (= 3-methylbut-2-enyl acetate), isoamyl acetate, dihydromyrcenol (2,6-dimethyloct-7-en-2-ol) and methylheptenone (6-methylhept-5-en-2-one) and mixtures thereof, and also mixtures thereof with one or more other aromas.

If trade names are specified above, these refer to the following sources:
¹ trade name of Symrise GmbH, Germany;
² trade name of Givaudan AG, Switzerland;
³ trade name of International Flavors & Fragrances Inc., USA;
⁴ trade name of BASF SE;
⁵ trade name of Danisco Seillans S.A., France;
⁹ trade name of Firmenich S.A., Switzerland;
¹⁰ trade name of PFW Aroma Chemicals B.V., the Netherlands.

The odorants include in particular compounds having a high volatility. These highly volatile odorant compounds include, but are not limited to the following compounds: rose oxide (tetrahydro-4-methyl-2-(2-methylpropenyl)-2H-pyran), 4-methyl-2-(2-methylpropyl)oxane or 4-methyl-2-(2-methylpropyl)-2H-pyran (Dihydrorosan®), prenyl acetate (= 3-methylbut-2-enyl acetate), isoamyl acetate, dihydromyrcenol (2,6-dimethyloct-7-en-2-ol) and methylheptenone (6-methylhept-5-en-2-one). If a mixture comprises at least one highly volatile odorant compound, the proportion of the highly volatile odorant compound is generally at least 1 % by weight, especially at least 5% by weight, for example 1 % to 99% by weight, especially 5% to 95% by weight, based on the total weight of the odorant composition.

Further odorants can be found, for example, in S. Arctander, Perfume and Flavor Chemicals, Vol. I and II, Montclair, N. J., 1969, Author’s edition or K. Bauer, D. Garbe and H. Surburg, Common Fragrance and Flavor Materials, 4th. Ed., Wiley-VCH, Weinheim 2001. Specifically, the following may be mentioned:

- extracts from natural raw materials such as essential oils, concretes, absolutes, resins, resinoids, balsams, tinctures, for example ambra tincture; amyris oil; angelica seed oil; angelica root oil; anise oil; valerian oil; basil oil; tree moss absolute; bay oil; mugwort oil; benzoin resin; bergamot oil; beeswax absolute; birch tar oil; bitter almond oil; savory oil; bucco leaf oil; cabreuva oil; cade oil; calamus oil; camphor oil; cananga oil; cardamom oil; cascarilla oil; cassia oil; cassie absolute; castoreum absolute; cedar leaf oil; cedar wood oil; cistus oil; citronella oil; lemon oil; copaiba balsam; copaiba balsam oil; coriander oil; costus root oil; cumin oil; cypress oil; davana oil; dill oil; dill seed oil; eau de brouts absolute; oakmoss absolute; elemi oil; estragon oil; eucalyptus citriodora oil; eucalyptus oil; fennel oil; spruce needle oil; galbanum oil; galbanum resin; geranium oil; grapefruit oil; guaiac wood oil; gurjun balsam; gurjun balsam oil; helichrysum absolute; helichrysum oil; ginger oil; iris root absolute; iris root oil; jasmine absolute; calamus oil; camellia oil blue; camellia oil roman; carrot seed oil; cascarilla oil; pine needle oil; spearmint oil; cumin oil; labdanum oil; labdanum absolute; labdanum resin; lavandin absolute; lavandin oil; lavender absolute; lavender oil; lemon grass oil; lovage oil; lime oil distilled; lime oil pressed; linalool oil; litsea cubeba oil; laurel leaf oil; macis oil; marjoram oil; mandarin oil; massoia bark oil; mimosa absolute; musk seed oil; musk tincture; clary sage oil; nutmeg oil; myrrh absolute; myrrh oil; myrtle oil; clove leaf oil; clove flower oil; neroli oil; olibanum absolute; olibanum oil; opopanax oil; orange blossom absolute; orange oil; oregano oil; palmarosa oil; patchouli oil; perilla oil; Peruvian balsam oil; parsley leaf oil; parsley seed oil; petitgrain oil; peppermint oil; pepper oil; allspice oil; pine oil; poley oil; rose absolute; rosewood oil; rose oil; rosemary oil; sage oil dalmatian; sage oil Spanish; sandalwood oil; celery seed oil; spike lavender oil; star anise oil; styrax oil; tagetes oil; fir needle oil; tea tree oil; turpentine oil; thyme oil; tolu balsam; tonka absolute; tuberose absolute; vanilla extract; violet leaf absolute; verbena oil; vetiver oil; juniper berry oil; wine yeast oil; vermouth oil; wintergreen oil; ylang oil; hyssop oil; civet absolute; cinnamon leaf oil; cinnamon bark oil; and fractions thereof or ingredients isolated therefrom;

Individual odorants are, for example, those from the group of
the hydrocarbons, for example 3-carene; alpha-pinene; beta-pinene; alpha-terpinene; gamma-terpinene; p-cymene; bisabolene; camphene; caryophyllene; cedrene; farnesene; limonene; longifolene; myrcene; ocimene; valencene; (E,Z)-1,3,5-undecatriene; styrene; diphenylmethane;
the aliphatic alcohols, for example hexanol; octanol; 3-octanol; 2,6-dimethylhep-tanol; 2-methyl-2-heptanol; 2-methyl-2-octanol; (E)-2-hexenol; (E)- and (Z)-3-hexenol; 1-octen-3-ol; mixture of 3,4,5,6,6-pentamethyl-¾-hepten-2-ol and 3,5,6,6-tetramethyl-4-methyleneheptan-2-ol; (E,Z)-2,6-nonadienol; 3,7-dimethyl-7-methoxyoctan-2-ol; 9-decenol; 10-undecenol; 4-methyl-3-decen-5-ol;
the aliphatic aldehydes and acetals thereof, for example hexanal; heptanal; oc-tanal; nonanal; decanal; undecanal; dodecanal; tridecanal; 2-methyloctanal; 2-methylnonanal; (E)-2-hexenal; (Z)-4-heptenal; 2,6-dimethyl-5-heptenal; 10-un-decenal; (E)-4-decenal; 2-dodecenal; 2,6,10-trimethyl-9-undecenal; 2,6,10-trimethyl-5,9-undecadienal; heptanal diethylacetal; 1,1-dimethoxy-2,2,5-trimethyl-4-hexene; citronellyloxyacetaldehyde; (E/Z)-1-(1-methoxypropoxy)-3-hexene; the aliphatic ketones and oximes thereof, for example 2-heptanone; 2-octanone; 3-octanone; 2-nonanone; 5-methyl-3-heptanone; 5-methyl-3-heptanone oxime; 2,4,4,7-tetramethyl-6-octen-3-one; 6-methyl-5-hepten-2-one;
the aliphatic sulfur-containing compounds, for example 3-methylthiohexanol; 3-methylthiohexyl acetate; 3-mercaptohexanol; 3-mercaptohexyl acetate; 3-mercaptohexyl butyrate; 3-acetylthiohexyl acetate; 1-menthene-8-thiol;
the aliphatic nitriles, for example 2-nonenenitrile; 2-undecenenitrile; 2-tridecene-nitrile; 3,12-tridecadienenitrile; 3,7-dimethyl-2,6-octadienenitrile; 3,7-dimethyl-6-octenenitrile;
the esters of aliphatic carboxylic acids, for example (E)- and (Z)-3-hexenyl formate; ethyl acetoacetate; isoamyl acetate; hexyl acetate; 3,5,5-trimethylhexyl acetate; 3-methyl-2-butenyl acetate; (E)-2-hexenyl acetate; (E)- and (Z)-3-hexenyl acetate; octyl acetate; 3-octyl acetate; 1-octen-3-yl acetate; ethyl butyrate; butyl butyrate; isoamyl butyrate; hexyl butyrate; (E)- and (Z)-3-hexenyl isobutyrate; hexyl crotonate; ethyl isovalerate; ethyl 2-methylpentanoate; ethyl hexanoate; allyl hexanoate; ethyl heptanoate; allyl heptanoate; ethyl octanoate; (E/Z)-ethyl 2,4-decadienoate; methyl 2-octynoate; methyl 2-nonynoate; allyl 2-isoamyloxyacetate; methyl 3,7-dimethyl-2,6-octadienoate; 4-methyl-2-pentyl crotonate;
the acyclic terpene alcohols, for example geraniol; nerol; linalool; lavandulol; nerolidol; farnesol; tetrahydrolinalool; 2,6-dimethyl-7-octen-2-ol; 2,6-dimethyl-octan-2-ol; 2-methyl-6-methylene-7-octen-2-ol; 2,6-dimethyl-5,7-octadien-2-ol; 2,6-dimethyl-3,5-octadien-2-ol; 3,7-dimethyl-4,6-octadien-3-ol; 3,7-dimethyl-1,5,7-octatrien-3-ol; 2,6-dimethyl-2,5,7-octatrien-1-ol; and the formates, acetates, propionates, isobutyrates, butyrates, isovalerates, pentanoates, hexanoates, crotonates, tiglinates and 3-methyl-2-butenoates thereof;
the acyclic terpene aldehydes and ketones, for example geranial; neral; citronellal; 7-hydroxy-3,7-dimethyloctanal; 7-methoxy-3,7-dimethyloctanal; 2,6,10-trimethyl-9-undecenal; geranyl acetone; and also the dimethyl and diethyl acetals of geranial, neral, 7-hydroxy-3,7-dimethyloctanal; the cyclic terpene alcohols, for example menthol; isopulegol; alpha-terpineol; terpineol-4; menthan-8-ol; men-than-1-ol; menthan-7-ol; borneol; isoborneol; linalool oxide; nopol; cedrol; ambrinol; vetiverol; guajol; and the formates, acetates, propionates, isobutyrates, butyrates, isovalerates, pentanoates, hexanoates, crotonates, tiglinates and 3-methyl-2-butenoates thereof;
the cyclic terpene aldehydes and ketones, for example menthone; isomenthone; 8-mercaptomenthan-3-one; carvone; camphor; fenchone; alpha-ionone; beta-ionone; alpha-n-methylionone; beta-n-methylionone; alpha-isomethylionone; beta-isomethylionone; alpha-irone; alpha-damascone; beta-damascone; beta-dama-scenone; delta-damascone; gamma-damascone; 1-(2,4,4-trimethyl-2-cyclohexen-1-yl)-2-buten-1-one; 1,3,4,6,7,8a-hexahydro-1,1,5,5-tetramethyl-2H-2,4a-meth-anonaphthalene-8(5H)-one; 2-methyl-4-(2,6,6-trimethyl-1-cyclohexen-1-yl)-2-bu-tenal; nootkatone; dihydronootkatone; 4,6,8-megastigmatrien-3-one; alpha-sinensal; beta-sinensal; acetylated cedar wood oil (methyl cedryl ketone);
the cyclic alcohols, for example 4-tert-butylcyclohexanol; 3,3,5-trimethylcyclohex-anol; 3-isocamphylcyclohexanol; 2,6,9-trimethyl-Z2,Z5,E9-cyclododecatrien-1-ol; 2-isobutyl-4-methyltetrahydro-2H-pyran-4-ol;
the cycloaliphatic alcohols, for example alpha-3,3-trimethylcyclohexylmethanol; 1-(4-isopropylcyclohexyl)ethanol; 2-methyl-4-(2,2,3-trimethyl-3-cyclopent-1-yl)butanol; 2-methyl-4-(2,2,3-trimethyl-3-cyclopent-1-yl)-2-buten-1-ol; 2-ethyl-4-(2,2,3-trimethyl-3-cyclopent-1-yl)-2-buten-1-ol; 3-methyl-5-(2,2,3-trimethyl-3-cyclopent-1-yl)pentan-2-ol; 3-methyl-5-(2,2,3-trimethyl-3-cyclopent-1-yl)-4-penten-2-ol; 3,3-dimethyl-5-(2,2,3-trimethyl-3-cyclopent-1-yl)-4-penten-2-ol; 1-(2,2,6-trimethylcyclohexyl)pentan-3-ol; 1-(2,2,6-trimethylcyclohexyl)hexan-3-ol;
the cyclic and cycloaliphatic ethers, for example cineol; cedryl methyl ether; cyclododecyl methyl ether; 1,1-dimethoxycyclododecane; 1,4-bis(ethoxymethyl)cyclohexane; (ethoxymethoxy)cyclododecane; alpha-cedrene epoxide; 3a,6,6,9a-tetramethyldodecahydronaphtho[2,1-b]furan; 3a-ethyl-6,6,9a-trimethyldodecahy-dronaphtho[2,1-b]furan; 1,5,9-trimethyl-13-oxabicyclo[10.1.0]trideca-4,8-diene; rose oxide; 2-(2,4-dimethyl-3-cyclohexen-1-yl)-5-methyl-5-(1-methylpropyl)-1,3-dioxane;
the cyclic and macrocyclic ketones, for example 4-tert-butylcyclohexanone; 2,2,5-trimethyl-5-pentylcyclopentanone; 2-heptylcyclopentanone; 2-pentylcyclo-pentanone; 2-hydroxy-3-methyl-2-cyclopenten-1-one; cis-3-methylpent-2-en-1-ylcyclopent-2-en-1-one; 3-methyl-2-pentyl-2-cyclopenten-1-one; 3-methyl-4-cy-clopentadecenone; 3-methyl-5-cyclopentadecenone; 3-methylcyclopentade-canone; 4-(1-ethoxyvinyl)-3,3,5,5-tetramethylcyclohexanone; 4-tert-pentylcyclo-hexanone; cyclohexadec-5-en-1-one; 6,7-dihydro-1,1,2,3,3-pentamethyl-4(5H)-indanone; 8-cyclohexadecen-1-one; 7-cyclohexadecen-1-one; (⅞)-cyclohexade-cen-1-one; 9-cycloheptadecen-1-one; cyclopentadecanone; cyclohexadecanone;
the cycloaliphatic aldehydes, for example 2,4-dimethyl-3-cyclohexenecarbalde-hyde; 2-methyl-4-(2,2,6-trimethylcyclohexen-1-yl)-2-butenal; 4-(4-hydroxy-4-methylpentyl)-3-cyclohexenecarbaldehyde; 4-(4-methyl-3-penten-1-yl)-3-cyclohexenecarbaldehyde;
the cycloaliphatic ketones, for example 1-(3,3-dimethylcyclohexyl)-4-penten-1-one; 2,2-dimethyl-1-(2,4-dimethyl-3-cyclohexen-1-yl)-1-propanone; 1-(5,5-dimethyl-1-cyclohexen-1-yl)-4-penten-1-one; 2,3,8,8-tetramethyl-1,2,3,4,5,6,7,8-octahydro-2-naphthalenyl methyl ketone; methyl 2,6,10-trimethyl-2,5,9-cyclododecatrienyl ketone; tert-butyl (2,4-dimethyl-3-cyclohexen-1-yl) ketone;
the esters of cyclic alcohols, for example 2-tert-butylcyclohexyl acetate; 4-tert-butylcyclohexyl acetate; 2-tert-pentylcyclohexyl acetate; 4-tert-pentylcyclohexyl acetate; 3,3,5-trimethylcyclohexyl acetate; decahydro-2-naphthyl acetate; 2-cyclo-pentylcyclopentyl crotonate; 3-pentyltetrahydro-2H-pyran-4-yl acetate; decahy-dro-2,5,5,8a-tetramethyl-2-naphthyl acetate; 4,7-methano-3a,4,5,6,7,7a-hexahy-dro-5- or -6-indenyl acetate; 4,7-methano-3a,4,5,6,7,7a-hexahydro-5- or -6-indenyl propionate; 4,7-methano-3a,4,5,6,7,7a-hexahydro-5- or -6-indenyl isobutyrate; 4,7-methanooctahydro-5- or -6-indenyl acetate;
the esters of cycloaliphatic alcohols, for example 1-cyclohexylethyl crotonate;
the esters of cycloaliphatic carboxylic acids, for example allyl 3-cyclohexylpropio-nate; allyl cyclohexyloxyacetate; cis- and trans-methyl dihydrojasmonate; cis- and trans-methyl jasmonate; methyl 2-hexyl-3-oxocyclopentanecarboxylate; ethyl 2-ethyl-6,6-dimethyl-2-cyclohexenecarboxylate; ethyl 2,3,6,6-tetramethyl-2-cyclohexenecarboxylate; ethyl 2-methyl-1,3-dioxolane-2-acetate;
the araliphatic alcohols, for example benzyl alcohol; 1-phenylethyl alcohol, 2-phenylethyl alcohol, 3-phenylpropanol; 2-phenylpropanol; 2-phenoxyethanol; 2,2-dimethyl-3-phenylpropanol; 2,2-dimethyl-3-(3-methylphenyl)propanol; 1,1-dimethyl-2-phenylethyl alcohol; 1,1-dimethyl-3-phenylpropanol; 1-ethyl-1-methyl-3-phenylpropanol; 2-methyl-5-phenylpentanol; 3-methyl-5-phenylpentanol; 3-phenyl-2-propen-1-ol; 4-methoxybenzyl alcohol; 1-(4-isopropylphenyl)ethanol;
the esters of araliphatic alcohols and aliphatic carboxylic acids, for example benzyl acetate; benzyl propionate; benzyl isobutyrate; benzyl isovalerate; 2-phenylethyl acetate; 2-phenylethyl propionate; 2-phenylethyl isobutyrate; 2-phenylethyl isovalerate; 1-phenylethyl acetate; alpha-trichloromethylbenzyl acetate; alpha,alpha-dimethylphenylethyl acetate; alpha,alpha-dimethylphenylethyl butyrate; cinnamyl acetate; 2-phenoxyethyl isobutyrate; 4-methoxybenzyl acetate;
the araliphatic ethers, for example 2-phenylethyl methyl ether; 2-phenylethyl isoamyl ether; 2-phenylethyl 1-ethoxyethyl ether; phenylacetaldehyde dimethyl acetal; phenylacetaldehyde diethyl acetal; hydratropaaldehyde dimethyl acetal; phenylacetaldehyde glycerol acetal; 2,4,6-trimethyl-4-phenyl-1,3-dioxane; 4,4a,5,9b-tetrahydroindeno[1,2-d]-m-dioxin; 4,4a,5,9b-tetrahydro-2,4-dimethylindeno[1,2-d]-m-dioxin;
the aromatic and araliphatic aldehydes, for example benzaldehyde; phenylacetaldehyde; 3-phenylpropanal; hydratropaaldehyde; 4-methylbenzaldehyde; 4-methylphenylacetaldehyde; 3-(4-ethylphenyl)-2,2-dimethylpropanal; 2-methyl-3-(4-isopropylphenyl)propanal; 2-methyl-3-(4-tert-butylphenyl) propanal; 2-methyl-3-(4-isobutylphenyl)propanal; 3-(4-tert-butylphenyl) propanal; cinnamaldehyde; alpha-butylcinnamaldehyde; alpha-amylcinnamaldehyde; alpha-hexylcinnamaldehyde; 3-methyl-5-phenylpentanal; 4-methoxybenzaldehyde; 4-hydroxy-3-methoxy-benzaldehyde; 4-hydroxy-3-ethoxybenzaldehyde; 3,4-methylenedioxybenzaldehyde; 3,4-dimethoxybenzaldehyde; 2-methyl-3-(4-methoxyphenyl)propanal; 2-methyl-3-(4-methylenedioxyphenyl) propanal;
the aromatic and araliphatic ketones, for example acetophenone; 4-methylacetophenone; 4-methoxyacetophenone; 4-tertbutyl-2,6-dimethylacetophenone; 4-phenyl-2-butanone; 4-(4-hydroxyphenyl)-2-butanone; 1-(2-naphthalenyl)ethanone; 2-benzofuranylethanone; (3-methyl-2-benzofuranyl) ethanone; benzophenone; 1,1,2,3,3,6-hexamethyl-5-indanyl methyl ketone; 6-tertbutyl-1,1-dimethyl-4-indanyl methyl ketone; 1-[2,3-dihydro-1,1,2,6-tetramethyl-3-(1-methylethyl)-1 H-5-indenyl]ethanone; 5',6',7',8'-tetrahydro-3',5',5',6',8',8'-hexamethyl-2-acetonaphthone;
the aromatic and araliphatic carboxylic acids and esters thereof, for example benzoic acid; phenylacetic acid; methyl benzoate; ethyl benzoate; hexyl benzoate; benzyl benzoate; methyl phenylacetate; ethyl phenylacetate; geranyl phenylacetate; phenylethyl phenylacetate; methyl cinnamate; ethyl cinnamate; benzyl cinnamate; phenylethyl cinnamate; cinnamyl cinnamate; allyl phenoxyacetate; methyl salicylate; isoamyl salicylate; hexyl salicylate; cyclohexyl salicylate; cis-3-hexenyl salicylate; benzyl salicylate; phenylethyl salicylate; methyl 2,4-dihydroxy-3,6-dimethylbenzoate; ethyl 3-phenylglycidate; ethyl 3-methyl-3-phenylglycidate;
the nitrogen-containing aromatic compounds, for example 2,4,6-trinitro-1,3-dimethyl-5-tert-butylbenzene; 3,5-dinitro-2,6-dimethyl-4-tert-butylacetophenone; cinna-monitrile; 3-methyl-5-phenyl-2-pentenonitrile; 3-methyl-5-phenylpentanonitrile; methyl anthranilate; methyl N-methylanthranilate; Schiffs bases of methyl anthranilate with 7-hydroxy-3,7-dimethyloctanal, 2-methyl-3-(4-tert-butylphenyl)propanal or 2,4-dimethyl-3-cyclohexenecarbaldehyde; 6-isopropylquinoline; 6-isobu-tylquinoline; 6-sec-butylquinoline; 2-(3-phenylpropyl)pyridine; indole; skatole; 2-methoxy-3-isopropylpyrazine; 2-isobutyl-3-methoxypyrazine;
the phenols, phenyl ethers and phenyl esters, for example estragole; anethole; eugenol; eugenyl methyl ether; isoeugenol; isoeugenyl methyl ether; thymol; car-vacrol; diphenyl ether; beta-naphthyl methyl ether; beta-naphthyl ethyl ether; beta-naphthyl isobutyl ether; 1,4-dimethoxybenzene; eugenyl acetate; 2-methoxy-4-methylphenol; 2-ethoxy-5-(1-propenyl)phenol; p-cresyl phenylacetate;
the heterocyclic compounds, for example 2,5-dimethyl-4-hydroxy-2H-furan-3-one; 2-ethyl-4-hydroxy-5-methyl-2H-furan-3-one; 3-hydroxy-2-methyl-4H-pyran-4-one; 2-ethyl-3-hydroxy-4 H-pyran-4-one;
the lactones, for example 1,4-octanolide; 3-methyl-1,4-octanolide; 1,4-nonanolide; 1,4-decanolide; 8-decen-1,4-olide; 1,4-undecanolide; 1,4-dodecanolide; 1,5-decanolide; 1,5-dodecanolide; 4-methyl-1,4-decanolide; 1,15-pentadecanolide; cis- and trans-11-pentadecen-1,15-olide; cis- and trans-12-pentadecen-1,15-olide; 1,16-hexadecanolide; 9-hexadecen-1,16-olide; 10-oxa-1,16-hexadecanolide; 11-oxa-1,16-hexadecanolide; 12-oxa-1,16-hexadecanolide; ethylene 1,12-dodecanedioate; ethylene 1,13-tridecanedioate; coumarin; 2,3-dihydrocoumarin; octahydrocoumarin.

In addition, suitable odorants are macrocyclic carbaldehyde compounds as described in WO 2016/050836.

Particular preference is given to mixtures of L-menthol and/or DL-menthol, L-menthone, L-menthyl acetate, or L-isopulegol, which are highly sought-after as analogs or substitutes for what are referred to as synthetic dementholized oils (DMOs). The mixtures of these minty compositions are preferably used in the ratio of L-menthol or DL-menthol 20-40% by weight, L-menthone 20-40% and L-menthyl acetate 0-20%, or in the ratio of 20-40% by weight, L-menthone 20-40% and L-isopulegol 0-20%.

The aforementioned odorant compounds and mixtures of odorant compounds can be used as such or in a solvent, which in itself is not an odorant. Typical solvents for odorant compounds are especially those having a boiling point at standard pressure above 150° C. and which do not dissolve organic polymers, e.g. diols, such as propanediol and dipropylene glycol, C₈-C₂₂ fatty acid C₁-C₁₀-alkyl esters, such as isopropyl myristate, di-C₆-C₁₀-alkyl ethers, e.g. dicapryl ether (Cetiol® OE from BASF SE), di-C₁-C₁₀-alkyl esters of aliphatic, aromatic or cycloaliphatic di- or tricarboxylic acids, for example dialkyl phthalates, such as dimethyl and diethyl phthalate and mixtures thereof, dialkyl hexa-hydrophthalates, e.g. dimethyl cyclohexane-1,2-dicarboxylate, diethyl cyclohexane-1,2-dicarboxylate and diisononyl 1,2-cyclohexanedicarboxylate, and dialkyl adipates, such as dibutyl adipate (e.g. Cetiol® B from BASF SE), C₈-C₂₂ fatty acid triglycerides, e.g. vegetable oils or cosmetic oils, such as octanoyl/decanoyltriglyceride (e.g. the commercial product Myritol® 318 from BASF SE), dimethyl sulfoxide and white oils.

The invention also relates to a device for controlled release of an odorant composition, where the device comprises a container for receiving the odorant composition, where the container has an opening, which is covered by an evaporation retardant membrane as described herein. This device is also termed “dispenser”.

The device of the present invention is typically a device for passive release of an odorant composition, which means that the release of the odorant composition exclusively occurs by diffusion of the odorant compounds through the evaporation retardant membrane.

The device of the present invention is usually analogue to the known dispensers for release of odorant compositions, which have a container for receiving the odorant composition and a membrane, which covers and thus closes the opening of the container, except for the fact that the membrane of the known devices is replaced by an evaporation retardant membrane. Suitable types of known dispensers, which can be modified by replacing a conventional membrane by an evaporation retardant membrane of the present invention are e.g. those described in DE 4205975, EP 489269, US 4,161,283, EP 1340513, EP 1728524, EP 1770870, WO 03/033039. In particular, the dispenser is an air freshener.

For example, the dispenser of the present invention may be simple pouch, where the wall material of the pouch is completely or partly an evaporation retardant membrane of the invention. The pouch also serves as the container for receiving the odorant composition. The dispenser of the present invention may also be a pouch formed by two or more interconnected foils, where one of the foils is an evaporation retardant membrane of the invention. These foils are typically welded or glued together to form a pouch or a flat dispenser.

The dispenser of the present invention may also be formed from (a) a deep-drawn plastic film made of a thermoplastic film material and featuring at least one zone designed, such as to form a container for receiving the odorant composition, and (b) the evaporation retardant membrane of the invention which is connected with the deep-drawn plastic film to form a durably sealed chamber which comprises the odorant composition.

In order to achieve as uniform release characteristics as possible, the bottom of the at least one zone is preferably designed such as to form a dish which is largely parallel with the membrane. Largely parallel means that at least 70% of the bottom area of the zone designed such as to form a dish is arranged in parallel with the membrane or forms an angle of less than 10° with the planar film.

Furthermore, it has proved advantageous when the depth of the zone designed such as to form a dish amounts to at least 0.2 mm, in particular to at least 0.4 mm and usually does not exceed 20 mm, in particular 10 mm. In the present context, depth is understood as meaning the distance of the bottom area of the dish and the plane predetermined by the edge of the dish, which are substantially plane-parallel relative to one another. The zone designed such as to form a dish will, as a rule, have an area in the range of from 2 to 50 cm².

The basic shape of the dispenser will typically be rectangular, it being possible for the corners to be rounded. In principle, however, oval, circular, triangular, trapezoid, diamond-shaped or polygonal embodiments with more than 4 sides are also possible. Preferably, the basic shape of the dispenser has characteristic dimensions (such as, for example, length of sides, diameter and the like) in the range of from 1 to 20 cm, in particular in the range of from 1.5 to 10 cm. In the case of a dispenser with a rectangular basic shape, the dimension of the one side of the rectangle will typically be in the range of from 2 to 20 cm, in particular in the range of from 2.5 to 10 cm, and the dimension of the side which is perpendicular thereto will be in the range of from 1 to 15 cm, in particular in the range of from 1.5 to 8 cm.

The dispenser may also feature a recess in the zone, where the membrane and the deep-drawn film connected. This recess will typically feature a circular, oval or polygonal area, whose edges are formed by the film material of the deep-drawn film and the membrane. The recess can also be provided with indentations or bulges to ensure better fastening. The extent of the recess along with a straight line through the center of the recess area will, as a rule, not be less than 3 mm and preferably not exceed 2 cm. The recess will typically have an area in the range of from 1 to 3 cm². This recess serves for attaching the dispenser. The recess is preferably oval or round, and may additionally be provided with indentations or bulges to ensure better fastening.

Typically, the deep-drawn film is less permeable for the odorant components than the evaporation retardant membrane by a factor of at least 10.

The deep-drawn film may consist of any thermoplastic processable plastic material. Examples of suitable plastic materials for the deep-drawn film are homo- and copolymers of C₂-C₆-olefins, in particular the homo- or copolymers of ethylene, and furthermore polyesters, polyvinyl chloride, polyvinylidene chloride, polyacrylonitrile, polystyrene, polycarbonate, polyethylene terephthalate, nylon, coextrudates of the abovementioned polymers, and laminates of the abovementioned polymers. As a rule, the deep-drawn plastic film has a thickness in the range of from 0.05 to 0.5 mm, in particular in the range of from 0.1 to 0.4 mm.

The deep-drawn film may comprise small amounts of stabilizers conventionally used for such polymers, for example antioxidants, which prevent or reduce aging of the plastic material. Such stabilizers may be present in the polymer in amounts of up to 1% by weight. Besides, the film material may also comprise customary amounts of processing aids, such as antiblocking agents and lubricant, for example erucamide or oleamide. These substances do not have any adverse effects on the properties of the dispenser.

The aforementioned dispensers can be easily prepared by i) providing a deep-drawn plastic film made of a thermoplastic polymer material, ii) filling the zone(s) designed such as to form a dish with the desired the odorant composition and iii) sealing the filled zone(s) designed such as to form a dish with the evaporation retardant membrane by connecting the evaporation retardant membrane with the deep-drawn plastic film.

To this end, one will, as a rule, in the first step i) form one or more dish-shaped zones (depressions) in the shape desired for the dispenser in a thermoplastically processable plastic film which is suitable for deep-drawing (deep-drawn film) by means of a customary deep-drawing method with heating and, optionally, with applying superatmospheric or subatmospheric pressure. In doing so, one will, as a rule, form the dish-shaped zones required for a plurality of, for example 2 to 100, in particular 4 to 20, dispensers in the deep-drawn film. Such processes are known to the skilled worker, for example from Saechtling, Kunststoff-Taschenbuch, 26th edition, Carl Hanser Verlag, Munich Vienna 1995, pp. 297-305 and G. Kühne in Kunststoff Maschinenführer (Johannaber, ed.), 3rd edition, Carl Hanser Verlag, Munich Vienna 1992, pp. 618-634.

Thereafter, the dish-shaped zone(s) formed thus is filled, in step ii), with the odorant composition, the amount of the odorant composition as a rule chosen such that it corresponds to the desired filling level.

In step iii), the filled zone(s) is (are) sealed with the evaporation retardant membrane. To this end, one will, as a rule, apply the evaporation retardant membrane to the deep-drawn film in such a way that the former covers the filled zones and subsequently fix it permanently, so that the filled zones are sealed durably. As a rule, fixing is effected by a welding method or a sealing method, where the evaporation retardant membrane on the deep-drawn film is durably bonded or sealed to the deep-drawn film. In principle, it is also possible to bond the evaporation retardant membrane together with the deep-drawn film. Such processes are known to the skilled worker, for example from Saechtling, Kunststoff-Taschenbuch, 26th edition, Carl Hanser Verlag, Munich Vienna 1995, pp. 305-325 and G. Kühne in Kunststoff Maschinenführer (Johannaber, ed.), 3rd edition, Carl Hanser Verlag, Munich Vienna 1992, pp. 747-810.

If the evaporation retardant membrane is connected to the deep-drawn film by a sealing method, it is necessary that the film material of the deep-drawn film is sealable with the material of the evaporation retardant membrane. It may, optionally, therefore be necessary that the deep-drawn film has a sealable coating which is sealable to the material of the evaporation retardant membrane. Sealable layers are well known to a skilled person and include low-molecular-weight homo- or copolymers of olefins, for example PE waxes as described in WO 2007/012621. The thickness of the sealable layer, if present, generally amounts to 5 to 100 µm, in particular to 10 to 80 µm and specifically to 15 to 50 µm.

In order to avoid a loss of odorant substance during storage, the dispenser may additionally comprise a protective layer, which completely covers the evaporation retardant membrane and is detachably attached thereto. The protective layer is impermeable for the components of the odorant composition and thus, the components of the odorant composition will be only released after the protective layer has been detached from the retardant membrane. The protective layer is typically a plastic foil, e.g. a polyamide foil or a polyvinyl alcohol foil, a metal foil or a metallized plastic foil.

The following examples are intended for illustration of the invention.

### 1) Abbreviations

pphm part by weight per hundred parts by weight of monomers
VP(25° C.) vapor pressure at 25° C.
Log Pow decadic logarithm of the partition coefficient between water and 1-octanol

### 2) Preparation of the Evaporation Retardant Membranes of the Present Invention

Starting materials:
i. Conventional uncoated paper 80 g/m2
ii. Polymer dispersion 1:
   Aqueous 47% by weight aqueous polymer dispersion prepared by emulsion polymerization of 55 pphm ethyl acrylate, 44 pphm methyl methacrylate and 1 pphm acrylic acid in the presence of 30 pphm degraded starch and 2 pphm of an anionic emulsifier as described in example 4 of WO 2013/083502.
iii. Polymer dispersion 2:
   Mixture of 9 parts of a 50% by weight aqueous polymer dispersion prepared by emulsion polymerization of 65 pphm styrene, 30 pphm butadiene, 3.5 pphm acrylic acid and 1.5 pphm acrylamide in the presence of an anionic emulsifier and 1 part of a 50% by weight nonionically stabilized aqueous paraffinic wax emulsion.

### Preparation Procedure

The two polymer dispersions were each printed on one half of the uncoated paper by flexographic printing in case of examples 1 and 2 or by rodcoating in case of comparative example C1. The coating thickness was 5 g/m², 10 g/m² or 15 g/m² as given in table 2. The coverage was 30%, 50%, 70% and 100% and is given in table 2. Uncoated paper was used as a further comparative C2.

Table 2

| Example | Polymer dispersion 1 | | Polymer dispersion 2 | |
|---|---|---|---|---|
| | Thickness | Coverage | Thickness | Coverage |
| 1 | 10 g/m² | 50% | 10 g/m² | 50% |
| 2 | 15 g/m² | 30% | 5 g/m² | 70% |
| V1 | 10 g/m² | 100% | 10 g/m² | 100% |
| V2 | | | | |

### 3) Test of the Evaporation Retardant Membranes of the Present Invention

The test was carried out according to the method described by Diehl, H. Pfeiffer, A.-M, Seyffer H., (2014), New testing method for functional barriers, International Paperworld IPW 8-11. All tests were carried out in a climate chamber at 23° C. and 50% humidity.

For this, circular specimens were cut from the paper coated with the respective coating. The specimen was placed in the holder of the evaporation cell. A defined amount of the respective volatile compound was filled into the chamber of the evaporation cell, and the chamber was closed by the holder and immediately weighed thereafter to an accuracy of 0.1 mg. The closed evaporation cell was reweighed after 1 h, 4 h, 1 d and 2 d. From these data, the loss of weight per day and square meter [g/(m²d)] was calculated.

The following volatile compounds were used for testing the barrier properties of the evaporation retardant membranes of the present invention:

Table 3

| | VP(25° C.) [mbar] | Log P_{ow} |
|---|---|---|
| n-Heptane | 61 | 4.8 |
| Limonene | 2 | 4.6 |
| n-Hexane | 201 | 3.9 |
| Citronellal | 0.5 | 3.8 |
| Isoamyl acetate | 5 | 2.5 |
| Hexanal | 15 | 1.9 |
| Isobutyl acetate | 20 | 1.8 |
| Ethyl acetate | 7 | 0.9 |

The results for each paper are given in the following tables 4 to 7.

Table 4

Weight loss of volatile compound through respective coating of paper 1

| | Polymer dispersion 1 | | Polymer dispersion 2 | |
|---|---|---|---|---|
| | Thickness 10 g/m² | Coverage 50% | Thickness 10 g/m² | Coverage 50% |
| | [g/(m²d)] | | [g/(m²d)] | |
| n-Heptane | 2 | | 1200 | |
| Limonene | 2 | | 100 | |
| n-Hexane | 4 | | 1900 | |
| Citronellal | 2 | | 2 | |
| Isoamyl acetate | 120 | | 204 | |
| Hexanal | 291 | | 702 | |
| Isobutyl acetate | 300 | | 320 | |
| Ethyl acetate | 1320 | | 1300 | |

Table 5

Weight loss of volatile compound through respective coating of paper 2

| | Polymer dispersion 1 | | Polymer dispersion 2 | |
|---|---|---|---|---|
| | Thickness 15 g/m² | Coverage 30% | Thickness 5 g/m² | Coverage 70% |
| | [g/(m²d)] | | [g/(m²d)] | |
| n-Heptane | 5 | | 603 | |
| Limonene | 5 | | 53 | |
| n-Hexane | 10 | | 953 | |
| Citronellal | 5 | | 4 | |
| Isoamyl acetate | 295 | | 103 | |
| Hexanal | 725 | | 353 | |
| Isobutyl acetate | 749 | | 163 | |
| Ethyl acetate | 3300 | | 653 | |

Table 6

Weight loss of volatile compound through respective coating of paper V1

| | Polymer dispersion 1 | | Polymer dispersion 2 | |
|---|---|---|---|---|
| | Thickness 10 g/m² | Coverage 100% | Thickness 10 g/m² | Coverage100% |
| | [g/(m²d)] | | [g/(m²d)] | |
| n-Heptane | 1 | | 600 | |
| Limonene | 1 | | 50 | |
| n-Hexane | 2 | | 950 | |
| Citronellal | 1 | | 1 | |
| Isoamyl acetate | 60 | | 100 | |
| Hexanal | 145 | | 350 | |
| Isobutyl acetate | 150 | | 160 | |
| Ethyl acetate | 660 | | 650 | |

Table 7

Weight loss of volatile compound through uncoated paper V2

| | No Coating |
|---|---|
| | [g/(m²d)] |
| n-Heptane | 4500 |
| Limonene | 1750 |
| n-Hexane | > 4000 |
| Citronellal | 950 |
| Isoamyl acetate | 2100 |
| Hexanal | 3750 |
| Isobutyl acetate | 3500 |
| Ethyl acetate | 2450 |

The data clearly show that the coating provides an evaporation retardant effect for all of the tested volatile compounds. The data also show that the evaporation retardant effect depends from the coverage and the polarity of the coating.

1-15. (canceled)

16. A sheet comprising a sheet-like support and at least one polymer coating arranged on at least one surface of the sheet-like support,
wherein the sheet-like support is permeable to fragrances and where on at least one surface of the sheet-like support at least one polymer coating is arranged only on a part of the surface of the sheet-like support, such that the total coverage of the surface by all polymer coatings is 10 to 90%, and each quarter square centimeter of said surface is covered by a polymer coating to an extent of at least 10%;
wherein the sheet is an evaporation retardant membrane for an odorant composition.

17. The sheet of claim 16, where each quarter square centimeter of said surface of the sheet-like support is covered by a polymer coating to an extent of 10 to 90%.

18. The sheet of claim 16, where the polymer coating is arranged on the surface of the sheet-like support as dots, dashes or as one or more grids or combinations thereof.

19. The sheet of claim 16, where at least two different polymer coatings are arranged on one surface of the sheet-like support, such that the coverage of the surface by the respective polymer coating is 5 to 85%, where the polymers of the two polymer coatings are in particular distinct from each other by their polarity and/or by their glass transition temperature.

20. The sheet of claim 19, where the different coatings are arranged on different areas of the surface of the sheet-like support, and where the total coverage of the respective area of the surface of the sheet-like support by the respective polymer coating is in particular from 10 to 90%.

21. The sheet of claim 16, where the total thickness of the polymer coating is in the range from 1 to 50 µm.

22. The sheet of claim 16, where the polymer coating is obtained by applying an aqueous polymer composition containing a polymer binder in the form of an aqueous polymer dispersion to the surface of the sheet-like support.

23. The sheet of claim 16, where the polymer of the polymer coating is selected from acrylate polymers.

24. The sheet of claim 16, where the sheet-like support is paper or cardboard.

25. The sheet of claim 16, where the aqueous polymer composition is applied to the surface of the sheet-like support by printing.

26. The sheet of claim 16, were the odorant composition comprises at least two different odorant compounds, where the different odorant compounds have different polarities and/or different vapor pressures.

27. A device for controlled release of an odorant composition, where the device comprises a container for receiving an odorant composition, where the container has an opening, which is covered by a membrane, which is the sheet of claim 16.

28. The device of claim 27, which additionally comprises a protective layer that completely covers the evaporation retardant membrane and is detachably attached thereto.

29. The device of claim 27, where the odorant composition contained in the container comprises at least two different odorant compounds, which have different polarities and/or different vapor pressures.

30. The use of a device of claim 27 for the controlled release of odorant compositions, which comprise at least two different odorant compounds having different polarities and/or different vapor pressures.
